SEQUENCE LISTING (15 sequences)

SEQ ID NO: 1 - RNA, 22 nt, Sus scrofa
  source 1..22: mol_type = other RNA
tccttcattccaccggagtctg

SEQ ID NO: 2 - RNA, 21 nt, Sus scrofa
  source 1..21: mol_type = other RNA
tcacagtgaaccggtctcttt

SEQ ID NO: 3 - RNA, 22 nt, Sus scrofa
  source 1..22: mol_type = other RNA
tagcagcacgtaaatattggcg

SEQ ID NO: 4 - RNA, 22 nt, Sus scrofa
  source 1..22: mol_type = other RNA
aaaccgttaccattactgagtt

SEQ ID NO: 5 - RNA, 22 nt, Sus scrofa
  source 1..22: mol_type = other RNA
taccctgtagaaccgaatttgt

SEQ ID NO: 6 - RNA, 22 nt, Sus scrofa
  source 1..22: mol_type = other RNA
cacccgtagaaccgaccttgcg

SEQ ID NO: 7 - RNA, 21 nt, Sus scrofa
  source 1..21: mol_type = other RNA
ctgacctatgaattgacagcc

SEQ ID NO: 8 - RNA, 22 nt, Sus scrofa
  source 1..22: mol_type = other RNA
cttatcaggttgtattgtaatt

SEQ ID NO: 9 - protein, 182 aa, Gossypium spp.
MGSLDSKESPANNPGLHSPPDEATKGYIMQQTMFRIKDPKPTLEFYSRVLGMSLLNKVDVPYMKMTLYMMGYEDVSSAPSDPVEKTIWTFGRPATMELTHFWGTENDPEFKGYHDGNSEPTGFGHIGITVDDMYKACERFESLGVEFVKKPGDGYAFIKDPDGYWIEIFDLNGIRAIVNNLA

SEQ ID NO: 10 - protein, 312 aa, synthetic construct
  REGION 1..312: note = AkrI protein
MRTTEISQGLKVSNLCIGAGPLGGMPTAFGYDVSEERGIATVQAVFDSPINFMDTSSEYGDSEVRIGKVIREGRMPKGFVIATKADAEGDGTARRGDFSAKQVRASFELSLERLGLTHIDVYFLHDPEKFPFEHVMARGGAMEELLRLKDEGLVGLVGVAGGDVVEMARYVDTGNLDILLNYGRYTLLDRSADSLIDKASDAGMAFLNAGPYASGLLAAPRGGSAWYQYAPPNADALQAVDQLHELCESFDVSLRSVALQFSMRDPRITSTVVGLSRPERVRALLDDAAAEIPAELWDQLPAPLNLPWNSLA

SEQ ID NO: 11 - protein, 312 aa, synthetic construct
  REGION 1..312: note = AkrII protein
MRTTEIAQGLKVSNLCIGAGPLGGMPTAFGYDVSEERGIATVQAVFDSPINFMDTSSEYGGSEARIGKVIREGLMPKGFVISSKADAEGEGVGRRGDFSAKQVRASFELSLERLGLTHLDVYFLHDPEKFPFEQVMARGGAMEELLRLKDEGLVGLVGVAGGDVLEMARYVDTGSLDILLNYGRYTLLDRSADSLITKVSDAGMAFLNAGPYASGLLAAPRGGSAWYQYAPPDATALQAVDQLHELCESFDVSLRSVALQFSMRDPRITSTVVGLSRPERVRALLDDEAAEIPAELWEQLPAPLNGDIVLPG

SEQ ID NO: 12 - protein, 312 aa, synthetic construct
  REGION 1..312: note = AkrIII protein
MRTTEIAQGLKVSNLCIGAGPLGGMPTAFGYDVSEERGIATVQAVFDSPINFMDTSSEYGGSEARIGKVIREGRMPKGFVISSKADAEGEGVGRRGDFSAKQVRASFELSLERLGLTHLDVYFLHDPEKFPFEQVMARGGAMEELLRLKDEGLVGLVGVAGGDVLEMARYVDTGSLDILLNYGRYTILDRSADSLITKVSDAGMAFLNAGPYASGLLAAPRGGSAWYQYAPPDATALQAVDQLHELCESFDVSLRSVALQFSMRDPRITSTVVGLSRPERVRALLDDEAAEIPAELWEQLPAPLNGDIVLPR

SEQ ID NO: 13 - protein, 252 aa, synthetic construct
  REGION 1..252: note = ADH-Lk protein
MTDRLKGKVAIVTGGTHGIGLAIATKFVTEGAKVVITGRRADVGEKAAKSIGGTDVIRFVQHDVSDEAGWGKLFDTTEETFGPVTTLVNNAGIGYRKSVEDTTTEEWRKLLSVNLDGVFFGTRLGIQRMKCKGLGASIINMSSILGQVGDPATGAYSASKGAVRIMSKSAALLCALKDYDVRVNTVHPGPIKTPLMDDMPGAEEMMSQRTKTPMGHIGEPNDIAWVCVYLASDESKFATGAEFVVDGGYTAQ

SEQ ID NO: 14 - protein, 333 aa, synthetic construct
  REGION 1..333: note = TAM-Ac protein
MAFSADTPEIVYTHDTGLDYITYSDYELDPANPLAGGAAWIEGAFVPPSEARISIFDQGFYTSDATYTTFHVWNGNAFRLGDHIERLFSNAESIRLIPPLTQDEVKEIALELVAKTELREAMVTVTITRGYSSTPFERDITKHRPQVYMSACPYQWIVPFDRIRDGVHLMVAQSVRRTPRSSIDPQVKNFQWGDLIRAIQETHDRGFELPLLLDCDNLLAEGPGFNVVVIKDGVVRSPGRAALPGITRKTVLEIAESLGHEAILADITPAELYDADEVLGCSTGGGVWPFVSVDGNSISDGVPGPVTQSIIRRYWELNVEPSSLLTPVQYALE

SEQ ID NO: 15 - protein, 608 aa, Pseudogluconobacter sp.
MRFEYLRQNVVGLALSTALIASLSGPAFAQHDANAAAEPSKAGQSAIENFQPVTADDLAGKNPANWPILRGNYQGWGYSPLDQINKDNVGDLQLVWSRTMEPGSNEGAAIAYNGVIFLGNTNDVIQAIDGKTGSLIWEYRRKLPSASKFINSLGAAKRSIALFGDKVYFVSWDNFVVALDAKTGKLAWETNRGQGVEEGVANSSGPIVVDGVVIAGSTCQFSGFGCYVTGTDAESGEELWRNTFIPRPGEEGDDTWGGAPYENRWMTGAWGQITYDPELDLVYYGSTGAGPASEVQRGTEGGTLAGTNTRFAVKPKTGEVVWKHQTLPRDNWDSECTFEMMVVSTSVNPDAKADGMMSVGANVPRGETRKVLTGVPCKTGVAWQFDAKTGDYFWSKATVEQNSIASIDDTGLVTVNEDMILKEPGKTYNYCPTFLGGRDWPSAGYLPKSNLYVIPLSNACYDVMARTTEATPADVYNTDATLVLAPGKTNMGRVDAIDLATGETKWSYETRAALYDPVLTTGGDLVFVGGIDRDFRALDAESGKEVWSTRLPGAVSGYTTSYSIDGRQYVAVVSGGSLGGPTFGPTTPDVDSASGANGIYVFALPEKK

(19) United States
(12) Patent Application Publication
(10) Pub. No.: US 2024/0368694 A1
(43) Pub. Date: Nov. 7, 2024

(54) BIOMARKER FOR TRICHOTHECENE TOXICITY

(71) Applicant: DSM Austria GmbH, Getzersdorf (AT)

(72) Inventors: Maia SEGURA-WANG, Tulln (AT); Veronika NAGL, Tulln (AT); Suzana ILIC, Tulln (AT); Bertrand GRENIER, Tulln (AT)

(21) Appl. No.: 18/683,116

(22) PCT Filed: Aug. 12, 2022
(86) PCT No.: PCT/EP2022/072650

§ 371 Date: Feb. 12, 2024

(30) Foreign Application Priority Data
Aug. 13, 2021 (EP) 21191318.1

(51) Int. Cl. C12Q 1/6883; C12Q 1/6851

(52) U.S. Cl. CPC C12Q 1/6883; C12Q 1/6851; C12Q 2600/142; C12Q 2600/178

(57) ABSTRACT

The present invention relates to a method for detecting a trichothecene effect comprising determining in a blood sample the expression level of at least one miRNA and comparing the expression level with a reference value, as well as the use of said at least miRNA for detecting a trichothecene effect in a blood sample.

### CROSS-REFERENCE TO RELATED APPLICATIONS

This application claims the benefit of priority of European patent application no. 21191318.1 filed 13 Aug. 2021, the content of which is being hereby incorporated by reference in their entirety for all purposes.

### FIELD OF THE INVENTION

The present invention relates to a novel method for detecting a trichothecene effect comprising determining the expression level of at least one miRNA in a test sample, as well as to a novel use of at least one miRNA for detecting a trichothecene effect in a test sample.

### BACKGROUND

Trichothecene mycotoxins are frequent contaminants of food and feed and cause adverse health effects in plants, animals and humans. The trichothecene deoxynivalenol (DON) is one of the economically most relevant mycotoxins worldwide. Among livestock species, pigs are regarded as the species most sensitive to the effects of DON and clinical symptoms include anorexia, emesis, weight loss or immuno-modulation. In humans, DON is associated with outbreaks of gastroenteritis. Consequently, regulatory limits in cereal-based food have been established for DON e.g., by the European Commission.

Although the general impact of DON on animal health is not disputable, there is a lack of toxicity markers that demonstrate the negative effects of DON in a reproducible manner. This is especially true for experiments employing realistic DON contamination levels in feed and minimal-or non-invasive sampling.

Already in 1996, Rotter et al. (Journal of Toxicology and Environmental Health—Part A, 48, 1-34) stated concerning DON that “the main overt effect at low dietary concentrations appears to be a reduction in food consumption”. As a direct result, the body weight of DON-induced pigs is reduced. However, this effect is not uniform, and the extent of body weight reduction underlies certain variations. Summarizing multiple feeding trials, the European Food Safety Authority (EFSA) concluded that doses starting from 0.6-2.0 ppm DON in feed decrease feed consumption and weight gain in pigs. Yet, recent studies showed that even exposure to 3 ppm DON does not necessarily result in significant effects on body weight in pigs (Grenier et al. Mol Nutr Food Res 55:761-771, 2011; Grenier et al., 2013, J Agric Food Chem 61:6711-6719). Considering the EU guidance levels for DON in swine feed (0.9 ppm; EC, 2006) and median DON occurrence in finished feed (0.3 ppm; Gruber-Dorninger et al., 2019, Toxins, 11, 375), it becomes evident that reduction of weight gain—although described as hallmark of DON toxicity—is not optimal to monitor the effects of this mycotoxin at practically relevant contamination levels.

Next to clinical signs and performance parameters, mechanism-based biomarkers can be used as tools to monitor the effects of mycotoxins. Baldwin et al. (2011, World Mycotoxin Journal, 4, 257-270) define mechanism-based biomarkers as “measurable biochemical or molecular indicators of biological response to a mycotoxin that can be specifically linked to the proximate cause. Mechanism-based biomarkers often include changes in the level of specific proteins, cellular metabolites, or gene expression resulting from specific alterations in metabolic or signaling pathways, stress responses, cell proliferation, or cell death.” As examples for DON mechanism-based biomarkers, authors list i) proinflammatory cytokines in blood or tissue, ii) up-regulation of hepatic SOC3 and mRNA/protein in tissue and iii) a decrease of IFGALS and IGF1 in blood. Cytokine levels in blood underlie marked time-dependent fluctuations, are therefore more routinely measured in tissues (mRNA level) in DON experiments. However, any measurement of biomarkers in tissue requires the euthanization of pigs, which is debatable due to animal welfare reasons. In addition, this clearly limits analysis on farm level and impedes experiments with multiple sampling time-points. Scarce reports on the effects of DON on blood cytokine levels provide contradictory results, e.g. Pasternak et al. (2018, Toxins, 10, 40) showed that 3.3-3.8 ppm DON in feed had no effect on serum interleukins IL1β, IL-8, IL-13, tumor necrosis factor or interferon-gamma in pigs. Only in 2017, EFSA re-evaluated available studies on the effects of DON on immunological parameters in pigs, concluding that “ . . . no suitable parameter for the risk assessment of DON (nor its acetylated and modified forms) could be identified among the various immune responses reported in these studies, nor could the observed size of the change of investigated immune responses be associated with relevant adverse immunological effects. Therefore, the CONTAM Panel concluded that no suitable concentration-response data on adverse immunological effects were available for the hazard characterization of pigs”. Concerning the effects of DON on serum levels of growth hormones, such as insulin like growth factor 1 (IGF1), a decrease has indeed been repeatably reported, albeit only at dietary DON concentrations≥3 ppm (Wang et al., 2019, Journal of agricultural and food chemistry, 67, 4976-4986; Wu et al., 2013, PLOS One, 8, e69502; Wu et al., 2015, BMC veterinary research, 11, 1-10).

Alternative approaches to indirectly assess the impact of DON on animal health comprise analysis of mycotoxin levels in feed and the measurement of exposure-based biomarkers.

Analysis of feed samples is accompanied by certain challenges. As summarized by Hennig-Pauka et al. (2018, Porcine health management, 4, 18), representative feed samples need to be collected, taking into account the non-homogeneous distribution of certain mycotoxins within a lot as well as time point of sampling, because feed lots might have exchanged between the time point of sampling and the initial onset of mycotoxin-induced effects.

The approach of exposure-based biomarkers describes the measurement of a mycotoxin (or its metabolite/s) in biological fluids (Baldwin et al., 2011, World Mycotoxin Journal, 4, 257-270). Those biomarkers are very good tools to study the toxicokinetics of DON or to assess the efficacy of mycotoxin-inactivating feed additives under controlled experimental conditions. However, as highlighted by Dänicke & Brezina (2013, Food and Chemical Toxicology, 60, 58-75), a correlation between exposure-based biomarkers for DON and clinical symptoms (or other health impairments induced by DON) is lacking so far. In addition, toxicokinetic limitations and analytical challenges hamper the application of those biomarkers on field.

There is thus a need to provide means and methods for detecting trichothecene mycotoxin exposure.

### SUMMARY

The present invention relates to a method for detecting a trichothecene effect comprising: (c) determining in a test sample that is a blood sample the expression level of at least one miRNA having at least 90% sequence identity, preferably having at least 95% sequence identity, preferably being identical to a sequence selected from the group consisting of SEQ ID NO: 1 (ssc-miR-205), SEQ ID NO: 2 (ssc-miR-128), SEQ ID NO: 3 (ssc-miR-16), SEQ ID NO: 4 (ssc-miR-451), SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p); and (d) comparing the expression level with a reference value.

The present invention also relates to a use of at least one miRNA having at least 90% sequence identity to a sequence selected from the group consisting of SEQ ID NO: 1 (ssc-miR-205), SEQ ID NO: 2 (ssc-miR-128), SEQ ID NO: 3 (ssc-miR-16), SEQ ID NO: 4 (ssc-miR-451), SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p) for detecting a trichothecene effect in a test sample that is a blood sample.

The present invention also relates to a data processing system comprising a processor configured to perform a method comprising the steps of (a) comparing the expression level of at least one miRNA having at least 90% sequence identity to a sequence selected from the group consisting of SEQ ID NO: 1 (ssc-miR-205), SEQ ID NO: 2 (ssc-miR-128), SEQ ID NO: 3 (ssc-miR-16), SEQ ID NO: 4 (ssc-miR-451), SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p) determined in a test sample that is a blood sample obtained from a subject with a reference value; (b) determining a deviation, wherein the reference value corresponds to the expression level that has been determined in a control sample that has been obtained from a subject that has not been exposed to a trichothecene; or determining no deviation, wherein the reference value corresponds to the expression level that has been determined in a control sample that has been obtained from a subject that has been exposed to a trichothecene; and (c) indicating whether a trichothecene effect is determined based on (b).

The present invention also relates to sequencing device capable of determining the level of at least one miRNA having at least 90% sequence identity to a sequence selected from the group consisting of SEQ ID NO: 1 (ssc-miR-205), SEQ ID NO: 2 (ssc-miR-128), SEQ ID NO: 3 (ssc-miR-16), SEQ ID NO: 4 (ssc-miR-451), SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p), comprising the data processing system of the invention.

The present invention also relates to a computer program comprising instructions to cause the data processing system of the invention or the sequencing device of the invention to execute the steps of (a) comparing the expression level of at least one miRNA having at least 90% sequence identity to a sequence selected from the group consisting of SEQ ID NO: 1 (ssc-miR-205), SEQ ID NO: 2 (ssc-miR-128), SEQ ID NO: 3 (ssc-miR-16), SEQ ID NO: 4 (ssc-miR-451), SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p) determined in a test sample that is a blood sample obtained from a subject with a reference value; (b) determining a deviation, wherein the reference value corresponds to the expression level that has been determined in a control sample that has been obtained from a subject that has not been exposed to a trichothecene; or determining no deviation, wherein the reference value corresponds to the expression level that has been determined in a control sample that has been obtained from a subject that has been exposed to a trichothecene; and (c) indicating whether a trichothecene effect is determined based on (b).

The present invention further relates to a computer-readable medium having stored thereon the computer program of the invention.

The present invention further relates to a kit for performing the method of the invention comprising a specific binding agent for at least one miRNA having at least 90% sequence identity to a sequence selected from the group consisting of SEQ ID NO: 1 (ssc-miR-205), SEQ ID NO: 2 (ssc-miR-128), SEQ ID NO: 3 (ssc-miR-16), SEQ ID NO: 4 (ssc-miR-451), SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p) comprised in or conjugated to a solid support

### BRIEF DESCRIPTION OF THE DRAWINGS

FIG. 1: Overview of trichothecenes.

FIG. 2: Chemical structure of DON and 7-one-8-hydroxy-8-ene-DON.

### DETAILED DESCRIPTION

The present invention provides a method for detecting a trichothecene effect, which method comprises determining in a test sample that is a blood sample the expression level of at least one miRNA described herein; and comparing the expression level with the expression level of said at least one miRNA in a control sample. miRNAs (also referred to as microRNAs) are short RNA molecules of a length of commonly from about 19 to about 24 nucleotides. miRNAs are negative regulators of gene expression capable of blocking translation of mRNA into proteins or of degrading mRNA molecules. By providing a method for detecting a trichothecene effect comprising determining in a blood sample the expression level of at least one miRNA described herein; and comparing the expression level with a reference value, the inventors have surprisingly identified a reliable way for detecting a trichothecene effect via a biomarker-of-effect which is suitable for application in the field.

The term “expression level” of a polynucleotide such as e.g. an mRNA molecule or a miRNA, refers to a relative or absolute amount of said polynucleotide. Expression levels of polynucleotides can be determined by appropriate strategies known to a person skilled in the art including but not limited to microarray experiments, quantitative real-time PCR (qPCR), or so-called next-generation sequencing (NGS) technologies offered e.g. by Illumina, Pacific Biosciences, Oxford Nanopore Technologies etc. Preferred methods for detecting miRNA expression are quantitative real-time PCR and sequencing methods, e.g. as essentially described in Example 1, with sequencing methods being most preferred.

Trichothecenes are a family of chemically related mycotoxins produced by various species of Fusarium, Myrothecium, Trichoderma, Trichothecium, Cephalosporium, Verticimonosporium, and Stachybotrys. Trichothecenes are a class of sesquiterpenes. The most important structural features causing the biological activities of trichothecenes are the 12,13-epoxy ring, the presence of hydroxyl or acetyl groups at appropriate positions on the trichothecene nucleus, and the structure and position of the side-chain. They are produced on many different grains like wheat, oats or maize by various Fusarium species such as F. graminearum, F. sporotrichioides, F. poae and F. equiseti.

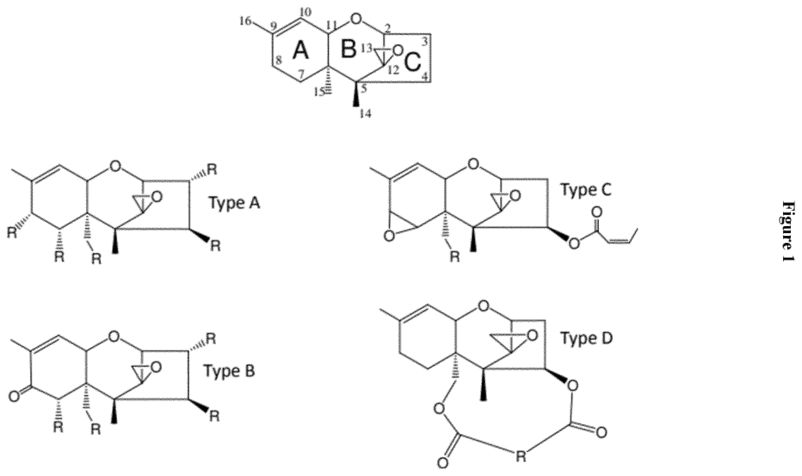

The family of trichothecene mycotoxins have been classified into four groups (types A, B, C, and D) based on the substitution pattern of EPT. Types A, B and C can be differentiated based on the substitution at the C-8 position (c.f. FIG. 1).

Type A trichothecenes include compounds that have a hydroxyl group at C-8 (e.g., neosolaniol), an ester function at C-8 (e.g., T-2 toxin), or no oxygen substitution at C-8 (e.g., trichodermin, 4,15-diacetoxyscirpenol, and harzianum A). Examples of type A trichothecenes are trichodermin (CAS no. 4682-50-2), scripentriol, 15-acetoxyscirpenol (CAS no. 2623-22-5), 4,15-diacetoxyscirpenol (CAS no. 2270-40-8), T-2 toxin (CAS no. 21259-20-1), HT-2 toxin (CAS no. 26934-87-2), T-2 tetraol (CAS no. 34114-99-3), trichodermin, isotrichodermin (CAS no. 91423-90-4), hydroxyisotrichodermin (CAS no. 344781-02-8) trichodermol (CAS no. 2198-93-8), calonectrin (CAS no. 38818-51-8), deacetylneosolaniol (CAS no. 74833-39-9), neosolaniol (CAS no. 36519-25-2), acetylneosolaniol (CAS no. 65041-92-1), sporotrichiol (CAS no. 101401-89-2).

Type B trichothecenes have a keto (carbonyl) function at C-8 (e.g., nivalenol, deoxynivalenol, and trichothecin). In Fusarium spp./genera, type B trichothecenes typically have a C-7 hydroxyl group, but this structural feature is not present in other genera. Examples of type B trichothecenes are nivalenol (CAS no. 23282-20-4), 15-acetyldeoxynivalenol (CAS no. 88337-96-6), fuseranon-X (CAS no. 23255-69-8), deoxynivalenol (CAS no. 51481-10-8), 3-keto deoxynivalenol, 3-epi deoxynivalenol, 3-amino deoxynivalenol.

Type C trichothecenes have a C-7/C-8 epoxide (e.g., crotocin).

Type D trichothecenes have an additional ring linking the C-4 and C-15 position (e.g., roridin A, verrucarin A, satratoxin H). Examples of type D trichothecenes are verrucarin A (CAS no. 3148-09-2), verrucarin J (CAS no. 4643-58-7).

In the context of the present disclosure, type B is the preferred class of trichothecenes. Within the class of type B trichothecenes, deoxynivalenol (DON) or deoxynivalenol derivatives are preferred, with DON being most preferred. DON and preferred DON derivatives are shown in the following table.

| Name | Abbreviation | Chemical | Formula | MW | CAS no |
|---|---|---|---|---|---|
| Deoxynivalenol | DON | (3α,7α)-3,7,15-trihydroxy-12,13- | C₁₅H₂₀O₆ | 296.3 | 51481-10-8 |
| | | epoxytrichothec-9-en-8-one | | | |
| 3-keto | 3K- | (7α)-7,15-dihydroxy-12,13- | C₁₅H₁₈O₆ | 294.3 | Not |
| deoxynivalenol | DON | epoxytrichothec-9-en-3,8-one | | | available |
| 3-epi | 3E- | (3β,7α)-3,7,15-trihydroxy-12,13- | C₁₅H₂₀O₆ | 296.3 | Not |
| deoxynivalenol | DON | epoxytrichothec-9-en-8-one | | | available |
| 15- | 15A- | 15-Acetyloxy-3α,7α-dihydroxy-12,13- | C₁₇H₂₂O₇ | 338.4 | 88337-96-6 |
| acetyldeoxynivalenol | DON | epoxytrichothec-9-en-8-one | | | |
| Nivalenol | NIV | (3α,4β,7α)-3,4,7,15-tetrahydroxy-12,13- | C₁₅H₂₀O₇ | 312.3 | 23282-20-4 |
| | | epoxytrichothec-9-en-8-one | | | |
| Fusarenon-X | FusX | (3β,4α,7α)-3,7,15-trihydroxy-8-oxo-12,13- | C₁₇H₂₂O₈ | 354.4 | 23255-69-8 |
| (4-acetylnivalenol) | | epoxytrichothec-9-en-4-yl acetate | | | |
| 3-amino | 3-amino | 3-amino-(7α)-7,15-dihydroxy-12,13- | C₁₅H₂₁NO₅ | 295.3 | Not |
| deoxynivalenol | DON | epoxytrichothec-9-en-8-one | | | available |
| 3-acyl | 3-acyl | (3α,7α)-3-(Acetyloxy)-12,13-epoxy-7,15- | C₁₇ H₂₂ O₇ | 338.4 | 50722-38-8 |
| deoxynivalenol | DON | dihydroxytrichothec-9-en-8-one | | | |

As used herein, the term “trichothecene effect” refers to exposure of a biological system to a trichothecene or a (physiological) effect of a trichothecene on a biological system. The biological system may be any biological system, such as a cell, a cell culture, a tissue, or an animal. A trichothecene can by any trichothecene disclosed herein, with a type B trichothecene being preferred. The type B trichothecene is preferably DON or a DON derivative, with DON being most preferred. The DON derivative is preferably one that is disclosed herein above. Preferably, “trichothecene effect” relates to trichothecene exposure.

A “test sample” as used herein preferably relates to a sample, for which it should be determined, whether or not the subject it has been obtained from has been exposed to a trichothecene. The test sample is a blood sample. The methods of the present invention may thus comprise step (b) providing the test sample.

The reference value may correspond to the expression level of said at least one miRNA according to the disclosure, that has been determined in one or more control samples. Depending on the type of the sample, such a control sample may be obtained from a subject that has been exposed to (a preferably known amount of) a trichothecene, or may have been obtained from a subject that has not been exposed to a trichothecene. Here, exposing a subject a trichothecene that is not bioavailable, e.g. because it is bound to another substance that prevents interaction of the trichothecene with a biological system, is equivalent to not exposing the subject to the trichothecene. The reference value may also be an average or mean value that has been determined in multiple control samples. The at least one miRNA according to the invention, may be selected from at least one miRNA having at least 90% sequence identity to a sequence selected from the group consisting of SEQ ID NO: 1 (ssc-miR-205), SEQ ID NO: 2 (ssc-miR-128), SEQ ID NO: 3 (ssc-miR-16), SEQ ID NO: 4 (ssc-miR-451), SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p). Preferably, the at least one miRNA is selected from at least one miRNA having at least 95% sequence identity to a sequence selected from the group consisting of SEQ ID NO: 1 (ssc-miR-205), SEQ ID NO: 2 (ssc-miR-128), SEQ ID NO: 3 (ssc-miR-16), SEQ ID NO: 4 (ssc-miR-451), SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p). More preferably the at least one miRNA is selected from the group consisting of SEQ ID NO: 1 (ssc-miR-205), SEQ ID NO: 2 (ssc-miR-128), SEQ ID NO: 3 (ssc-miR-16), SEQ ID NO: 4 (ssc-miR-451), SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p). The at least one miRNA of the invention may also be a combination of two, three, four, five, six, seven, or all eight miRNAs selected from aforementioned group, with a combination of two being preferred. Hereby, a trichothecene effect can be determined with particularly high reliability.

The miRNA can be a miRNA, wherein an increase in the expression level in comparison with the reference value is indicative for a trichothecene effect. Such a miRNA may be a miRNA selected from a miRNA having at least 90% sequence identity to a sequence selected from the group consisting of SEQ ID NO: 1 (ssc-miR-205), SEQ ID NO: 2 (ssc-miR-128), SEQ ID NO: 3 (ssc-miR-16), and SEQ ID NO: 4 (ssc-miR-451). Preferably, such a miRNA is a miRNA selected from a miRNA having at least 95% sequence identity to a sequence selected from the group consisting of SEQ ID NO: 1 (ssc-miR-205), SEQ ID NO: 2 (ssc-miR-128), SEQ ID NO: 3 (ssc-miR-16), and SEQ ID NO: 4 (ssc-miR-451). More preferably, such a miRNA is a miRNA selected from the group consisting of SEQ ID NO: 1 (ssc-miR-205), SEQ ID NO: 2 (ssc-miR-128), SEQ ID NO: 3 (ssc-miR-16), and SEQ ID NO: 4 (ssc-miR-451). In the context of the present invention a miRNA, wherein an increase in the expression level in comparison with the reference value is indicative for a trichothecene effect, is particularly preferred.

The miRNA can be a miRNA, wherein a decrease in the expression level in comparison with the reference value is indicative for a trichothecene effect. Such a miRNA may be a miRNA selected from a miRNA having at least 90% sequence identity to a sequence selected from the group consisting of SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p). Preferably, such a miRNA is a miRNA selected from a miRNA having at least 95% sequence identity to a sequence selected from the group consisting of SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p). More preferably, such a miRNA is a miRNA selected from the group consisting of SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p).

Preferably, the at least one miRNA according is selected from the group consisting of a miRNA having at least 90% sequence identity to SEQ ID NO: 1 (ssc-miR-205) and a miRNA having at least 90% sequence identity to SEQ ID NO: 2 (ssc-miR-128). More preferably, the at least one miRNA according is selected from the group consisting of a miRNA having at least 95% sequence identity to SEQ ID NO: 1 (ssc-miR-205) and a miRNA having at least 95% sequence identity to SEQ ID NO: 2 (ssc-miR-128). Even more preferably, the at least one miRNA according is selected from the group consisting of SEQ ID NO: 1 (ssc-miR-205) and SEQ ID NO: 2 (ssc-miR-128).

Accordingly, the at least one miRNA referred to herein preferably comprises a miRNA having at least 90% sequence identity, preferably having at least 95% sequence identity, preferably being identical to SEQ ID NO: 1 (ssc-miR-205).

Alternatively or additionally, the at least one miRNA referred to herein preferably comprises a miRNA having at least 90% sequence identity, preferably having at least 95% sequence identity, preferably being identical to SEQ ID NO: 2 (ssc-miR-128).

Throughout the application, unless specified otherwise, miRNAs are denoted by accession numbers of the miRBase sequence database in the version of release 21 of June 2014 (Kozomara A, Griffiths-Jones S. Nucleic Acids Res. 2014 42:D68-D73; Kozomara A, Griffiths-Jones S. Nucleic Acids Res. 2011 39:D152-D157; Griffiths-Jones S, Saini H K, van Dongen S, Enright A J. Nucleic Acids Res. 2008 36:D154-D158; Griffiths-Jones S, Grocock R J, van Dongen S, Bateman A, Enright A J. Nucleic Acids Res. 2006 34:D140-D144; Griffiths-Jones S. Nucleic Acids Res. 2004 32:D109-D111). The following table summarizes some of the miRNAs of the disclosure.

| | | SEQ |
|---|---|---|
| Accession No. | Sequence | ID NO |
| ssc-miR-205 | UCCUUCAUUCCACCGGAGUCUG | 01 |
| ssc-miR-128 | UCACAGUGAACCGGUCUCUUU | 02 |
| ssc-miR-16 | UAGCAGCACGUAAAUAUUGGCG | 03 |
| ssc-miR-451 | AAACCGUUACCAUUACUGAGUU | 04 |
| ssc-miR-10b | UACCCUGUAGAACCGAAUUUGU | 05 |
| ssc-miR-99b | CACCCGUAGAACCGACCUUGCG | 06 |
| ssc-miR-192 | CUGACCUAUGAAUUGACAGCC | 07 |
| ssc-miR-374a-3p | CUUAUCAGGUUGUAUUGUAAUU | 08 |

Comparing the level of said at least one miRNA can be based on comparing the individual values of one or more miRNA. For example, a deviation in the expression levels of a miRNA between the test sample and reference value may be indicative for a trichothecene effect (such as a trichothecene exposure), in particular if the reference value corresponds to the expression level of said miRNA in a control sample obtained from a subject that has not been exposed to a trichothecene. A deviation in the expression levels may be indicative for a trichothecene exposure. The deviation may be expressed in a normal scale, or preferably in a log-fold scale, such as log 2-fold scale. The deviation is preferably statistically significant. Several techniques for testing the statistical significance of results are known to the skilled person and it is well within the skill of the person skilled in the art to choose and apply the most appropriate method for analyzing statistical significance. Statistical significance may e.g. be expressed by a p-value or a FDR p adjusted value and the significance level may be ≤0.1, preferably ≤0.07, or more preferably ≤0.005. The deviation may be an increase or decrease. A deviation indicating a trichothecene effect may e.g. be at least about 1.3-fold, more preferably at least about 1.5-fold and most preferably at least about 2-fold compared to a reference value corresponding to a sample obtained from a subject not having been exposed to a trichothecene.

Alternatively, where the reference value corresponds to the expression level that has been determined in a control sample that has been obtained from a subject that has been exposed to a trichothecene, a non-deviation, a non-significant deviation, or an (untypically) low deviation between the values of test sample and the reference value may be indicative for a trichothecene effect, such as a trichothecene exposure.

The term “detect” or “detecting” when used herein in the context of detecting a trichothecene effect may generally refer to quantitative or qualitative detection. For example, “detect” may refer to qualitative detection, i.e. whether or not a trichothecene effect is present based on the comparison step. “Detect” may also refer to a quantitative detection, i.e. to the extent of the trichothecene effect based on the comparison step.

A “sample” as used herein refers to a blood sample. The sample may have been obtained from a subject. A blood sample includes, but is not limited to, whole blood, serum, plasma, or a sample comprising blood cells, such as a peripheral blood mononuclear cell (PBMC) sample, with blood serum samples being preferred. A blood sample allows a rapid detection of a trichothecene effect in a subject and thus achieve maximum applicability in the field, because a sample that can be obtained with little effort. By providing a method wherein the sample is a blood (serum) sample, a potential trichothecene effect can be assessed most conveniently. A blood serum sample can be obtained e.g. by punctuation of the Vena cava cranialis or the Vena jugalaris. Serum samples can be stored at −80° C. for later analysis.

Comparing the level of said at least one miRNA can also be based on comparing the individual values of multiple miRNAs. Here the relative (or less preferred the absolute) deviations in the expression level in the test sample and the reference value may be combined to yield an even more significant result than comparing the level of only one miRNA. Alternatively, deviation values may be multiplied with or divided by each other, depending on the expected direction of deviation in case of a trichothecene effect.

Comparing the level of said at least one miRNA to a reference value, in particular if multiple miRNAs are involved, can also be carried out using complex methods, such as computer-assisted methods, in particular methods involving machine learning. As an illustrative example, an artificial neural network system can be used for comparing the expression level(s) with a reference value. The neural network may be trained by being provided with the value of one or more reference values that may correspond to miRNA levels from one or more samples. Preferably, both, reference values for a trichothecene effect and reference values for no trichothecene effect may be used for training the neural network. The trained neural network may then be used to detect a trichothecene effect. For this purpose, the values of miRNA level(s) of respective miRNA(s) of a test sample may be used as input for the neural network, and the neural network may provide an indication of a trichothecene effect, such as a qualitative information, a quantitative information, a score, a probability or any other type of information useful in the detection or characterization of a trichothecene effect. Methods of constructing, training, and applying a neural network are well known to the skilled person. Other (computer-assisted) approaches may also be suitable for being applied in the present invention and are also known to the skilled person, such as e.g. Bayesian networks or decision trees.

Generally, the outcome of the comparing step (d) may be any type of information indicating whether or not a trichothecene effect has been detected, such as whether or not the subject the sample has been obtained from has been exposed to a trichothecene effect. The method may thus further comprise step (e): detecting a trichothecene effect if the expression level of said at least one miRNA in the test sample deviates in comparison with the reference value.

The information may be a qualitative information, a quantitative information, a score, a probability or any other type of useful information. The method of the present invention may thus comprise the further step of providing information regarding a trichothecene effect, i.e. providing information on the test result. The information may be provided by any suitable mean known to the skilled person. Suitable means include standard communication methods such as by mail, telephone, telefax, electronic message (such as email), internet-based communication, social media, etc.

A “subject” as used herein refers to a vertebrate, preferably a mammal, an avian, or a fish. The term “mammal” as used herein refers to any animal classified as a mammal, including, without limitation, humans, pigs, cows, horses, dogs, or primates such as cynomolgus monkeys, to name only a few illustrative examples. A subject can be a domestic animal, a farm animal, a zoo animal, a sport animal, and/or a pet animal, with a farm animal being preferred. Preferred subjects include a pig, a human, a cow, a horse, a dog, a chicken, a fish, in particular puffer, and/or a catfish. The subject is preferably not a mouse. A preferred subject is of the genus Sus, preferably of the species Sus scrofa. Another preferred subject is a human.

Generally, a subject may be any subject, that expresses one or more miRNA according to the disclosure. Such subjects includes Ateles geoffroyi (Geoffroy's spider monkey), Homo sapiens, Gorilla gorilla, Lagothrix lagotricha (brown woolly monkey), Macaca mulatta (rhesus macaque, rhesus monkey), Macaca nemestrina (southern pig-tailed macaque), Pan paniscus (bonobo), Pongo pygmaeus (Bornean orangutan), Pan troglodytes (chimpanzee), Saguinus labiatus (white-lipped tamarin), Sus scrofa (pig), Bos taurus (cow), Equus caballus (horse), Canis familiaris (domestic dog), Cyprinus carpio (common carp or European carp), Danio rerio (zebrafish, fish), Fugu rubripes (Japanese puffer), Ictalurus punctatus (channel catfish), Tetraodon nigroviridis, Gallus gallus (chicken). Further, less important subjects include Cricetulus griseus (Chinese hamster), Ornithorhynchus anatinus (platypus), Monodelphis domestica (gray short-tailed opossum), Mus musculus (mouse), Rattus norvegicus (brown rat), Sarcophilus harrisii (Tasmanian devil), Xenopus laevis (African clawed frog), and Xenopus tropicalis (western clawed frog).

A trichothecene effect, in particular exposure to a trichothecene, can manifest itself as adverse health effects, such as anorexia, emesis, weight loss or immuno-modulation, especially in mammalian subjects and in a particularly dramatical manner in subjects of the genus Sus. In humans, trichothecene is associated with outbreaks of gastroenteritis. Methods of the present invention may thus be for diagnosis, in particular diagnosis of a trichothecene exposure of the subject. Such methods may have the purpose of determining a medical condition of a subject in order to decide whether or not or what type of measure should be taken in order to maintain or improve the health of the subject.

Methods of the present invention may also be non-diagnostic. As an illustrative example, a non-diagnostic method can be a feed sampling method. Feed sampling is usually not very accurate due to the non-homogenous distribution of mycotoxins in the feed and typical sample sizes used for this analysis. Due to the comparably large amount of feed uptake by a subject, such as a pig, determining a trichothecene exposure of the subject may give a more reliable test result of whether or not the feed is contaminated with a trichothecene than analyzing the feed directly. Methods of the present invention may therefore be methods for detection of a trichothecene in feed or food. Methods of the present invention may comprise prior to obtaining the test sample from the subject step (a) feeding the subject with feed or food suspected to contain a trichothecene.

To avoid the undesirable consequences of prolonged exposure to a trichothecene, it may become necessary to treat a trichothecene contaminated feed or food with a trichothecene neutralizing agent or to apply a trichothecene neutralizing method, in case a trichothecene effect is detected in a subject. Therefore, the present invention further relates to a method as described above, wherein the method is for selecting food or feed for contacting with a trichothecene neutralizing agent or for applying a trichothecene neutralizing method. By applying the method described for selecting food or feed for contacting with a trichothecene neutralizing agent or for applying a trichothecene neutralizing method, adverse health effects can be at least mitigated or completely avoided. Methods of the present invention may thus comprise step (a) feeding the subject with feed suspected to contain a trichothecene prior to obtaining the test sample from the subject. Methods of the present invention may also comprise step (f) selecting the feed for contacting with a trichothecene neutralizing agent or applying a trichothecene neutralizing method if a deviation in the expression levels of the at least one miRNA between the test sample and the control sample indicates a trichothecene effect. Methods of the present invention may thus comprise providing a trichothecene neutralizing agent or applying a trichothecene neutralizing method. Methods of the present invention may also comprise contacting the feed or food selected by the method with the trichothecene neutralizing agent or applying the trichothecene neutralizing method to the feed.

A trichothecene neutralizing agent or method may refer to an agent or method that converts a trichothecene, e.g. by altering its molecular structure, which can be the effected by chemical, enzymatic, or physical interaction with the trichothecene neutralizing agent. A less toxic molecule may be the result of such a treatment. Thus, neutralization of a trichothecene can be by conversion. The term “conversion”, as used herein includes isomerization, hydrolysis, derivatization, conjugation, and degradation. A trichothecene neutralizing agent or method may also reduce bioavailability of the trichothecene, e.g. making the trichothecene unavailable to the subject, preferably to the subject's digestion system. This may be achieved, for example by an agent that binds or adsorbs the trichothecene. In general, a trichothecene neutralizing agent may be any suitable type of agent, which may be selected from the group consisting of one or more polypeptide(s), one or more microorganism(s), and one or more trichothecene-binding agent(s). Thus, neutralization of a trichothecene can also be by binding.

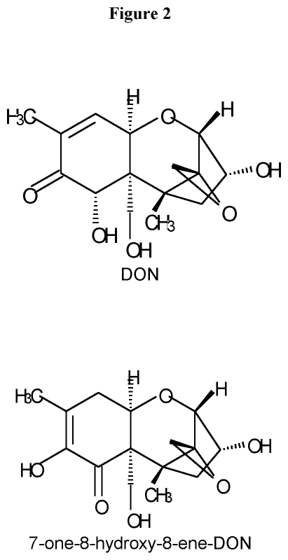

A trichothecene neutralizing agent may be an enzyme that is capable of modifying the C7-atom of the trichothecene, which is preferably DON or a DON derivative having a hydroxy group at C7 and a carbonyl group at C8, and preferably no substitution at C10, following the atom numbering proposed by Yoshizawa and Morooka in the first description of DON (Yoshizama and Morooka (1973), Agric. Biol. Chem. 37, 2933-2934). The term “modifying the C7-atom” may refer to a process of intramolecular rearrangement and/or change of bond/s and/or binding partner/s and/or functional group/s at C7 atom of the carbon chain (e.g., position 7 of the carbon chain), e.g., of DON and/or DON derivative/s, (e.g., comprising changing (e.g., oxidizing)) a hydroxyl-moiety (i.e., —OH) to carbonyl moiety (e.g., ═O) at the C7-atom (i.e., position 7 of the carbon chain) and/or causing a chemical change, e.g., breaking bond/s and/or making new bond/s. A trichothecene neutralizing agent may be a polypeptide comprising the amino acid sequence set forth in SEQ ID NO: 9, or a polypeptide comprising an amino acid sequence having at least 70% (e.g., at least 80%, at least 85%, at least 90%, at least 91%, at least 92%, at least 93%, at least 94%, at least 95%, at least 96%, at least 97%, at least 98%, at least 99%) identity to the amino acid sequence set forth in SEQ ID NO: 9. The polypeptide SEQ ID NO: 9 is capable of converting DON to 7-one-8-hydroxy-8-ene-DON (cf. FIG. 2). Trichothecene neutralizing agents are further described in EP 20203359.3, which is incorporated by reference.

The sequence of SEQ ID NO: 9 is shown in the Table below.

| SEQ ID NO | Sequence |
|---|---|
| 9 | MGSLDSKESPANNPGLHSPPDEATKGYIMQQTMFRIKDPKPTLEFYSRVLGMS |
| | LLNKVDVPYMKMTLYMMGYEDVSSAPSDPVEKTIWTFGRPATMELTHFWGT |
| | ENDPEFKGYHDGNSEPTGFGHIGITVDDMYKACERFESLGVEFVKKPGDGYAF |
| | IKDPDGYWIEIFDLNGIRAIVNNLA |

The relatedness between two amino acid sequences or between two nucleotide sequences (e.g. DNA and RNA like miRNA) is described by the parameter “identity” or “sequence identity”.

For purposes of the present invention, the sequence identity between two polypeptide or nucleotide sequences (e.g. DNA, RNA, miRNA) is preferably determined using the Needleman-Wunsch algorithm (Needleman, Wunsch. J Mol Biol. 1970. 48(3):443-453). The output of Needle labeled “identity” (obtained using the no-brief option) is used as the percent identity and is calculated as follows: (Identical Residues×100)/(Length of Alignment−Total Number of Gaps in Alignment). Appropriate tools are available online, e.g. from NCBI (National Center for Biotechnology Information; e.g. https://blast.ncbi.nlm.nih.gov/Blast.cgi?PAGE_TYPE=BlastSearch&PROG_DEF=blastn&BLAST_PROG_DEF=blastn&BLAST_SPEC=GlobalAln&LINK_LOC=BlastHomeLink) using default algorithm parameters for amino acid sequences (proteins) and nucleotide sequences. It is considered that a global alignment algorithm such as the Clustal Omega tool from the European Bioinformatics Institute shall be used (e.g. https://www.ebi.ac.uk/Tools/msa/clustalo/) using the default settings for protein input sequences for amino acid sequence alignments, for DNA input sequences for DNA alignments and for RNA for RNA or miRNA alignments is preferred.

Those having skill in the art will know how to determine percent identity between/among sequences using algorithms as known in the art.

A trichothecene neutralizing agent may also be a compound that catalyzes a biochemical reaction that is a modification of the C8-atom of a type B trichothecene, preferably DON and/or DON-derivative/s. Preferably wherein (i) said modifying is a transformation, e.g. substitution or the change of a functional group of a chemical species (e.g., carbonyl-, keto-, and/or oxo-moiety (═O)) of the C8-atom of the type B trichothecene, preferably DON or DON derivative, into a different functional group (e.g., a hydroxyl (—OH) or amino (—NH2) moiety); and/or (ii) said modifying is modifying of only the C8-atom of the type B trichothecene, preferably DON or DON derivative/s; and/or (iii) said modifying consists of modifying the C8-atom of the type B trichothecene, preferably DON or DON derivative/s; and/or (iv) said modifying is not comprising molecular rearrangement, e.g., not comprising a change in which there is a bond migration of an atom or bond and/or an entering group takes up a different position from the leaving group, with accompanying bond migration; and/or (v) said modifying is not comprising modifying of the C7-atom of the type B trichothecene, preferably DON or DON derivative/s; and/or (vi) said modifying is not comprising isomerization (e.g., intramolecular isomerization) of the type B trichothecene, preferably DON or DON derivative/s, e.g., wherein the product of said modifying is isomeric with a corresponding reactant, e.g., the type B trichothecene, preferably DON or DON derivative. Exemplary compounds are shown in the following table.

| SEQ ID NO | Sequence |
|---|---|
| 10 | MRTTEISQGLKVSNLCIGAGPLGGMPTAFGYDVSEERGIATVQAVFDSPINFMD |
| | TSSEYGDSEVRIGKVIREGRMPKGFVIATKADAEGDGTARRGDFSAKQVRASF |
| | ELSLERLGLTHIDVYFLHDPEKFPFEHVMARGGAMEELLRLKDEGLVGLVGVA |
| | GGDVVEMARYVDTGNLDILLNYGRYTLLDRSADSLIDKASDAGMAFLNAGPY |
| | ASGLLAAPRGGSAWYQYAPPNADALQAVDQLHELCESFDVSLRSVALQFSMR |
| | DPRITSTVVGLSRPERVRALLDDAAAEIPAELWDQLPAPLNLPWNSLA |
| 11 | MRTTEIAQGLKVSNLCIGAGPLGGMPTAFGYDVSEERGIATVQAVFDSPINFM |
| | DTSSEYGGSEARIGKVIREGLMPKGFVISSKADAEGEGVGRRGDFSAKQVRAS |
| | FELSLERLGLTHLDVYFLHDPEKFPFEQVMARGGAMEELLRLKDEGLVGLVG |
| | VAGGDVLEMARYVDTGSLDILLNYGRYTLLDRSADSLITKVSDAGMAFLNAG |
| | PYASGLLAAPRGGSAWYQYAPPDATALQAVDQLHELCESFDVSLRSVALQFS |
| | MRDPRITSTVVGLSRPERVRALLDDEAAEIPAELWEQLPAPLNGDIVLPG |
| 12 | MRTTEIAQGLKVSNLCIGAGPLGGMPTAFGYDVSEERGIATVQAVFDSPINFM |
| | DTSSEYGGSEARIGKVIREGRMPKGFVISSKADAEGEGVGRRGDFSAKQVRAS |
| | FELSLERLGLTHLDVYFLHDPEKFPFEQVMARGGAMEELLRLKDEGLVGLVG |
| | VAGGDVLEMARYVDTGSLDILLNYGRYTILDRSADSLITKVSDAGMAFLNAGP |
| | YASGLLAAPRGGSAWYQYAPPDATALQAVDQLHELCESFDVSLRSVALQFSM |
| | RDPRITSTVVGLSRPERVRALLDDEAAEIPAELWEQLPAPLNGDIVLPR |
| 13 | MTDRLKGKVAIVTGGTHGIGLAIATKFVTEGAKVVITGRRADVGEKAAKSIGG |
| | TDVIRFVQHDVSDEAGWGKLFDTTEETFGPVTTLVNNAGIGYRKSVEDTTTEE |
| | WRKLLSVNLDGVFFGTRLGIQRMKCKGLGASIINMSSILGQVGDPATGAYSAS |
| | KGAVRIMSKSAALLCALKDYDVRVNTVHPGPIKTPLMDDMPGAEEMMSQRT |
| | KTPMGHIGEPNDIAWVCVYLASDESKFATGAEFVVDGGYTAQ |
| 14 | MAFSADTPEIVYTHDTGLDYITYSDYELDPANPLAGGAAWIEGAFVPPSEARISI |
| | FDQGFYTSDATYTTFHVWNGNAFRLGDHIERLFSNAESIRLIPPLTQDEVKEIAL |
| | ELVAKTELREAMVTVTITRGYSSTPFERDITKHRPQVYMSACPYQWIVPFDRIR |
| | DGVHLMVAQSVRRTPRSSIDPQVKNFQWGDLIRAIQETHDRGFELPLLLDCDN |
| | LLAEGPGFNVVVIKDGVVRSPGRAALPGITRKTVLEIAESLGHEAILADITPAEL |
| | YDADEVLGCSTGGGVWPFVSVDGNSISDGVPGPVTQSIIRRYWELNVEPSSLLT |
| | PVQYALE |

The compound that catalyzes such a biochemical reaction can be an Akr variant, which is a cofactor-dependent oxidoreductase. Cofactors can be e.g. NADPH or NADH or other typical enzyme cofactors that are known to those skilled in the art.

An Akr I polypeptide may comprise the sequence of SEQ ID NO: 10. An Akr I polypeptide (variant) may comprise one or more equivalent (or same) amino acid/s in position/s corresponding to: G18, A19, G20, D54, Y59, K84, H125, D126, A160, G161, Y182, A209, G210, P211, Y212, A213, S214, G215, R255, V272, V273, G274, L275, R280, A283, L284, particularly preferred D54, Y59, K84 and/or H125 position/s of SEQ ID NO: 10. An Akr I polypeptide (variant) may comprise an amino acid sequence having at least 70% (e.g., at least 80%, at least 85%, at least 90%, at least 91%, at least 92%, at least 93%, at least 94%, at least 95%, at least 96%, at least 97%, at least 98%, at least 99%) identity to the amino acid sequence set forth in SEQ ID NO: 10.

An Akr II polypeptide may comprise the sequence of SEQ ID NO: 11. An Akr II polypeptide (variant) may comprise one or more equivalent (or same) amino acid/s in position/s corresponding to: G18, A19, G20, D54, Y59, K84, H125, D126, A160, G161, Y182, A209, G210, P211, Y212, A213, S214, G215, R255, V272, V273, G274, L275, R280, A283, L284, particularly preferred D54, Y59, K84 and/or H125 position/s of SEQ ID NO: 11. An Akr II polypeptide (variant) may comprise an amino acid sequence having at least 70% (e.g., at least 80%, at least 85%, at least 90%, at least 91%, at least 92%, at least 93%, at least 94%, at least 95%, at least 96%, at least 97%, at least 98%, at least 99%) identity to the amino acid sequence set forth in SEQ ID NO: 11.

An Akr III polypeptide may comprise the sequence of SEQ ID NO: 12. An Akr III polypeptide (variant) may comprise one or more equivalent (or same) amino acid/s in position/s corresponding to: G18, A19, G20, D54, Y59, K84, H125, D126, A160, G161, Y182, A209, G210, P211, Y212, A213, S214, G215, R255, V272, V273, G274, L275, R280, A283, L284, particularly preferred D54, Y59, K84 and/or H125 position/s of SEQ ID NO: 12. An Akr III polypeptide (variant) may comprise an amino acid sequence having at least 70% (e.g., at least 80%, at least 85%, at least 90%, at least 91%, at least 92%, at least 93%, at least 94%, at least 95%, at least 96%, at least 97%, at least 98%, at least 99%) identity to the amino acid sequence set forth in SEQ ID NO: 12.

A trichothecene neutralizing agent may also be an ADH-Lk variant (polypeptide) (e.g., SEQ ID NO. 13). Such an enzyme may catalyze the reduction of DON to 3,7,8R,15-tetrahydroxyscirpine (THS). The ADH-Lk polypeptide (variant) may comprise one or more equivalent (or same) amino acid/s in position/s corresponding to: N114, S143, Y156 and/or K160 position/s of SEQ ID NO: 13; and/or G14, H17, G18, I19, G20, G38, R39, R40, H62, D63, V64, N90, A91, G92, 193, V113, M141, S142, S143, Y156, K160, P188, G189, P190, I191, T193, P194 and/or L195 position/s of SEQ ID NO: 13; and/or E67, V99, E100, T102, T104, W107, R108, L111, L115, D116, F119, F120, T122, R123, 1126, Q127, K130, G146, Q147, G149, D150, P151, G154, S157, A158, G161, A162, R164, 1165, M166, K168, S169, A170, L172, L173 and/or C174 position/s of SEQ ID NO: 13; and/or R4, E67, V99, E100, T102, T104, W107, R108, L111, L115, D116, F119, F120, T122, R123, I126, Q127, K130, G146, Q147, V148, G149, D150, P151, G154, S157, A158, G161, A162, R164, 1165, M166, K168, S169, A170, A171, L172, L173, C174, A175, P190, T212, P213, M214, G215, H216, I217, G218, E219, D222, W225, V226, Y229, E234, K236, F237, A238, T239, G240, A241, E242, F243, V244, V245, D246, G247, G248, Y249, T250, A251 and/or Q252 position/s of SEQ ID NO: 13. An ADH-Lk polypeptide (variant) may comprise an amino acid sequence having at least 70% (e.g., at least 80%, at least 85%, at least 90%, at least 91%, at least 92%, at least 93%, at least 94%, at least 95%, at least 96%, at least 97%, at least 98%, at least 99%) identity to the amino acid sequence set forth in SEQ ID NO: 13.

A trichothecene neutralizing agent may also be a compound that catalyzes transamination of DON. Transaminases (EC 2.6.1.-) catalyze the reversible transfer of an amino group from an amine to a carbonyl compound (acceptor). Transamination of DON and/or its derivatives with additional carbonyl groups, such as 3-keto DON to aminated metabolites (e.g., compounds or intermediates, such as for example 8-amino-3-keto DON) catalyzed by, e.g., SEQ ID NO. 14 (TAM-Ac). A TAM-Ac polypeptide (variant) may comprise one or more equivalent (or same) amino acid/s in position/s corresponding to: K188, Y67, R86, K188, E221, 1246, T247, T283, R86, K188, E221, G59, F60, T62, S63, A65, Y67, S93, T124, T126, Y148, F190, D194, 1196, Q200 and/or D214 position/s of SEQ ID NO: 14. A TAM-Ac polypeptide (variant) may comprise an amino acid sequence having at least 70% (e.g., at least 80%, at least 85%, at least 90%, at least 91%, at least 92%, at least 93%, at least 94%, at least 95%, at least 96%, at least 97%, at least 98%, at least 99%) identity to the amino acid sequence set forth in SEQ ID NO: 14.

Exemplary Akr (variants), ADH-Lk (variants), and TAM-Ac (variants) are described in EP20199475.3, which is incorporated herewith by reference.

A trichothecene neutralizing agent may also be an alcohol dehydrogenase, e.g. as shown in SEQ ID NO: 15. Suitable alcohol dehydrogenases are described in EP3273802A1, which is incorporated herewith by reference.

| SEQ ID NO | Sequence |
|---|---|
| 15 | MRFEYLRQNVVGLALSTALIASLSGPAFAQHDANAAAEPSKAGQSAIENFQPV |
| | TADDLAGKNPANWPILRGNYQGWGYSPLDQINKDNVGDLQLVWSRTMEPGS |
| | NEGAAIAYNGVIFLGNTNDVIQAIDGKTGSLIWEYRRKLPSASKFINSLGAAKR |
| | SIALFGDKVYFVSWDNFVVALDAKTGKLAWETNRGQGVEEGVANSSGPIVVD |
| | GVVIAGSTCQFSGFGCYVTGTDAESGEELWRNTFIPRPGEEGDDTWGGAPYEN |
| | RWMTGAWGQITYDPELDLVYYGSTGAGPASEVQRGTEGGTLAGTNTRFAVK |
| | PKTGEVVWKHQTLPRDNWDSECTFEMMVVSTSVNPDAKADGMMSVGANVP |
| | RGETRKVLTGVPCKTGVAWQFDAKTGDYFWSKATVEQNSIASIDDTGLVTVN |
| | EDMILKEPGKTYNYCPTFLGGRDWPSAGYLPKSNLYVIPLSNACYDVMARTTE |
| | ATPADVYNTDATLVLAPGKTNMGRVDAIDLATGETKWSYETRAAL YDPVLTT |
| | GGDLVFVGGIDRDFRALDAESGKEVWSTRLPGAVSGYTTSYSIDGRQYVAVV |
| | SGGSLGGPTFGPTTPDVDSASGANGIYVFALPEKK |

A trichothecene neutralizing agent may also be a microorganism that is capable of converting a trichothecene. Suitable microorganisms can be from the genus Eubacterium (or meanwhile reclassified as Raoultibacter), such as BBSH797, DSM11798. Such microorganisms are described in EP 1042449 B1, which is incorporated herewith by reference. Further, a suitable microorganism may be Eggerthella sp. DII-9 (reclassified to Raoultibacter sp. DII-9). Such microorganisms are described in CN 106119177 A, which is incorporated herewith by reference. A further suitable microorganism may be Slackia sp. D-G6 as described by Gao et al., 2020, Toxins 2020, 12, 85, which is incorporated herewith by reference.

As described above, a trichothecene neutralizing agent may also reduce bioavailability of the trichothecene e.g. by binding and/or adsorption. Examples for such agents can include cholestyramine, divinylbenzene-styrene, bentonites, montmorillonite-rich clays, zeolites, organoclays, activated charcoal, cellulose, yeast or yeast cell wall-derived products.

Trichothecene neutralizing methods may comprise but are not limited to methods of contacting a trichothecene neutralizing agent with the feed. Methods may also comprise heat treatment or ultrasonic treatment, mechanical sorting and separation, density segregation.

The assessment of the efficacy and/or efficiency of mycotoxin neutralizing agents or mycotoxin neutralizing methods poses a considerable challenge due to the lack of suitable methods to allow a determination of whether or not the applied mycotoxin neutralizing agent or method achieved the desired the effect. Methods of the present invention may thus also serve the purpose of assessing the capacity of a method or (test) compound to neutralize a trichothecene. With other words a method of the invention may be for assessment of the capacity of a method or compound to neutralize a trichothecene. Such a compound may be selected from the group consisting of one or more polypeptide(s), one or more microorganism(s), including live, dead, lyophilized, autolyzed and/or dormant microorganism(s), and one or more trichothecene-binding agent(s), including organic and/or an organic trichothecene-binding agent(s). By using the methods of the present invention, the assessment of the efficacy and/or efficiency of a trichothecene neutralizing agent or method can be achieved in a particularly fast and quick manner.

In cases where a sample has been obtained from a subject, the method of the invention may thus comprise prior to obtaining the test sample from the subject step (a3) feeding the feed contacted with a trichothecene and the test compound to the subject. In such a case, the method may further comprise steps of contacting the feed with a trichothecene, contacting the feed with the test compound, and/or contacting a trichothecene with the test compound. In general, such steps can be performed in any order. Accordingly the method may comprise prior to step (a3) steps of: (a1) contacting the feed with a trichothecene, and (a2) contacting the feed contacted with a trichothecene with the test compound; or (a1′) contacting the feed with the test compound, and (a2′) contacting the feed contacted with the test compound with a trichothecene; or (a1″) contacting a trichothecene with the test compound, and (a2″) contacting the trichothecene contacted with the test compound with the feed.

In some cases, the reference value is obtained from a control sample that is obtained from a subject that has been fed with a control feed. The control sample used in such a method may be a negative control sample or positive control sample. A negative control sample used in this context refers to a sample obtained from a subject that has been fed with a control feed that is essentially free of trichothecenes, or a control feed that has been contacted with a trichothecene and a compound that neutralizes the trichothecene. In such a method, the absence of a deviation or a non-significant deviation in the expression levels between the test sample and the positive control sample may be indicative for the capacity of the test compound to neutralize a trichothecene. A positive control sample used in this context refers to a sample obtained from a subject that has been fed with a control feed that has been contacted with a trichothecene. In such a case, the presence of a (preferably significant) deviation in the expression levels between the test sample and the positive control sample may be indicative for the capacity of the test compound to neutralize a trichothecene.

The present invention also relates to a use of at least one miRNA according to the invention for detecting a trichothecene effect in a test sample. The at least one miRNA is a miRNA having at least 90% sequence identity, preferably having at least 95% sequence identity, preferably being identical to a sequence selected from the group consisting of SEQ ID NO: 1 (ssc-miR-205), SEQ ID NO: 2 (ssc-miR-128), SEQ ID NO: 3 (ssc-miR-16), SEQ ID NO: 4 (ssc-miR-451), SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p). Preferably, the at least one miRNA according to the invention may be selected from the group consisting of SEQ ID NO: 1 (ssc-miR-205) and SEQ ID NO: 2 (ssc-miR-128). The use may be in any method of the invention. Such a use may be for the purpose of detecting a trichothecene exposure of a subject, assessing the capacity of a test compound or a method to neutralize a trichothecene, or selecting a feed for treatment with a trichothecene neutralizing agent or method. By using at least one miRNA as described herein for detecting a trichothecene effect in a test sample, a trichothecene effect can be detected with especially high reliability and specificity.

To allow a rapid reaction such as e.g. the supply with a counteracting agent in case of a trichothecene exposure, fast availability of test results is desired. This can for example be achieved if determination of the expression level of at least one miRNA in a test sample and comparing the expression level with a reference value is performed with an integrated system. The methods of the invention may thus be computer-implemented methods, which may be carried out by an apparatus for carrying out such a method.

Thus, the present invention also relates to a data processing system comprising a processor configured to perform a method comprising the steps of comparing the expression level of at least one miRNA according to the invention determined in a test sample obtained from a subject with a reference value. Input regarding the expression level of the at least one miRNA is required. The information may be transmitted to the data processing system via a device that is capable of determining such information, e.g. over some transmission medium, such as over electrical wiring or cabling, through fiber optics, or via electromagnetic radiation. Such a device may be integrated in the data processing system or connected to it. Information regarding the expression level of the at least one miRNA may also be transmitted to the data processing system via input through a user interface. If the reference value corresponds to the expression level of said miRNA in a control sample that has been obtained from a subject that has not been exposed to a trichothecene, a (significant) deviation in the expression levels of a miRNA between the test sample and reference value may be indicative for a trichothecene effect (such as a trichothecene exposure). In such a case, the method may comprise indicating a trichothecene effect of the subject if a deviation is determined. Alternatively, if the reference value corresponds to the expression level that has been determined is a control sample that has been obtained from a subject that has been exposed to a trichothecene, no deviation between the values of test sample and the reference value may be indicative for a trichothecene effect, such as a trichothecene exposure. In such a case, the method may comprise indicating a trichothecene effect of the subject if no deviation or no significant deviation is determined. “No deviation” in this context may also encompass a non-significant deviation, or an (untypically) low deviation. The at least one miRNA may be a mi RNA having at least 90% sequence identity, preferably having at least 95% sequence identity, preferably being identical to a sequence selected from the group consisting of SEQ ID NO: 1 (ssc-miR-205), SEQ ID NO: 2 (ssc-miR-128), SEQ ID NO: 3 (ssc-miR-16), SEQ ID NO: 4 (ssc-miR-451), SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p). Preferably, the at least one miRNA according to the invention may be selected from the group consisting of SEQ ID NO: 1 (ssc-miR-205) and SEQ ID NO: 2 (ssc-miR-128).

Ideally, the data processing system is connected to or integrated in a device that is capable of determining the expression level of at least one miRNA according to the invention in a test sample. The device may provide the data processing system with information regarding the expression level of said at least one miRNA. Such a device may for example be a sequencing device, such as a quantitative real-time sequencing device. Sequencing devices are known to the skilled person and are e.g. commercially available by Illumina, Pacific Biosciences, Oxford Nanopore Technologies etc. Particularly useful are portable sequencing devices such as a MinION sequencing (Oxford Nanopore Technologies). A device that is capable of determining the expression level of at least one miRNA according to the invention may also be a microarray reader.

Accordingly, the present invention also relates to a sequencing device capable of determining the level of at least one miRNA according to the present invention, comprising the data processing system of the invention. The at least one miRNA may be a miRNA having at least 90% sequence identity, preferably having at least 95% sequence identity, preferably being identical to a sequence selected from the group consisting of SEQ ID NO: 1 (ssc-miR-205), SEQ ID NO: 2 (ssc-miR-128), SEQ ID NO: 3 (ssc-miR-16), SEQ ID NO: 4 (ssc-miR-451), SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p). Preferably, the at least one miRNA according to the invention may be selected from the group consisting of SEQ ID NO: 1 (ssc-miR-205) and SEQ ID NO: 2 (ssc-miR-128).

The present invention also relates to a computer program containing instructions to cause the data processing system disclosed herein or the sequencing device disclosed herein to execute the steps of (a) comparing the expression level of at least one miRNA according to the invention determined in a test sample obtained from a subject with a reference value, (b) determining a deviation, wherein the reference value corresponds to the expression level that has been determined in a control sample that has been obtained from a subject that has not been exposed to a trichothecene; or determining no deviation, wherein the reference value corresponds to the expression level that has been determined in a control sample that has been obtained from a subject that has been exposed to a trichothecene; and (c) indicating a trichothecene effect of the subject if a deviation is determined, wherein reference value corresponds to the expression level that has been determined in a control sample that has been obtained from a subject that has not been exposed to a trichothecene; or indicating a trichothecene effect of the subject if no deviation is determined, wherein the reference value corresponds to the expression level that has been determined in a control sample that has been obtained from a subject that has been exposed to a trichothecene. When the computer program is loaded into and executed by a data processing system, the data processing system becomes an apparatus for carrying out the methods described herein.

The computer program can be a computer program product accessible from a computer-usable or computer-readable medium providing program code for use by or in connection with a computer or any instruction execution system. The present invention thus also relates to a computer-readable medium having stored thereon the computer program disclosed herein. A computer-usable or computer readable medium can be any apparatus that may include, store, communicate, propagate, or transport the program for use by or in connection with the instruction execution system, apparatus, or device. The medium can be an electronic, magnetic, optical, electromagnetic, infrared, semiconductor, or paper system (or apparatus or device) or a propagation medium. The medium may be any suitable tangible medium, such as a diskette, a CD, a DVD, a BD, a hard drive, a memory stick, a memory card or any other computer readable storage medium.

In order to achieve particularly cost-effective and fast detection of a trichothecene effect, a kit is provided for performing the method of detecting a trichothecene effect in the sense of the present invention, comprising a specific binding agent for at least one miRNA of the invention comprised on or conjugated to a solid support. The at least one miRNA may be a miRNA having at least 90% sequence identity, preferably having at least 95% sequence identity, preferably being identical to a sequence selected from the group consisting of SEQ ID NO: 1 (ssc-miR-205), SEQ ID NO: 2 (ssc-miR-128), SEQ ID NO: 3 (ssc-miR-16), SEQ ID NO: 4 (ssc-miR-451), SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p). Preferably, the at least one miRNA according to the invention may be selected from the group consisting of SEQ ID NO: 1 (ssc-miR-205), SEQ ID NO: 2 (ssc-miR-128). The kit may comprise an unlabeled specific binding partner and a labeled specific binding partner for the at least one miRNA. Preferably, the unlabeled specific binding partner is conjugated to the solid support. The kit may comprise specific binding partners for no more than 1, 2, 3, 4, 5, 6, 7, 8, 9, 10, 11, 12, 13, 14, 15, 16, 17, 18, 19, 20, 21, 22, or 23 miRNAs. In some applications, the kit may comprise specific binding partners for no more than 24, 48, 96, or 384 miRNAs. The kit may comprise specific binding partners for no more than 1, 2, 3, 4, 5, 6, 7, or 8 miRNAs according to methods of the invention. It is particularly preferred that the kit comprises specific binding partners for up to 8 different miRNA, wherein the up to 8 different miRNA is preferably selected from the group consisting of SEQ ID NOs 1-8, with SEQ ID NOs 1 and 2 being preferred and SEQ ID NO: 1 being most preferred. The kit may only comprise specific binding partners for miRNAs according to the methods of the invention.

Such a kit may be for a lateral flow test. The kit may comprise a lateral flow device (LFD), which comprises a carrier (solid support), which has at least two zones, a conjugate pad and a test zone. The conjugate pad comprises a labeled binding agent for the at least one miRNA according to the invention, that can be mobilized in a solvent (e.g. a solvent that comprises target molecule(s)) and be transported therein towards the test zone. The test zone comprises an un-labelled binding agent that is also capable of binding a target molecule. Hereby, targets (the at least one miRNA) that are also bound by the labelled agent from the conjugate pad are immobilized in the test zone. An optional additional control zone may comprise another immobilized binding agent, capable of binding the labelled binding agent from the conjugate pad which was not retained in the test zone. Examples of lateral flow devices are disclosed in Koczula and Gallotta. Essays Biochem. 2016 Jun. 30;60(1):111-20 or Krska and Molinelli. Anal Bioanal Chem. 2009 January;393(1):67-71. Accordingly, the kit disclosed herein may comprise a lateral flow assay kit.

Also, immunochemical detection could be done by ELISA, e.g. Sandwich-ELISA. The kit may thus comprise a (preferably unlabeled) specific binding agent for the target miRNA that can be immobilized on a solid support or is immobilized on a solid support. The test sample may be added on to the support. Further, a labelled binding agent for the target miRNAs can be added. The miRNA-bound label may then be used for quantification of the miRNA. Accordingly, the kit disclosed herein may comprise an ELISA assay kit.

The specific binding agent may for example be a nucleic acid, an antibody, an antigen binding fragment of an antibody, or a proteinaceous molecule having antibody-like properties. Where both, a labeled and an unlabeled specific binding partner, are used in the kit, it is preferred that both binding partners can simultaneously bind to the target miRNA. This may for example be achieved if one specific binding partner can bind (e.g. hybridize) to one part of the target miRNA, while the other specific binding partner can bind (e.g. hybridize) to another part of the target miRNA.

The kit may further comprise means for detecting a miRNA that is bound to a specific binding agent, such as an unlabeled specific binding agent described herein, which is preferably conjugated to a solid support. Such means may comprise a probe conjugated to a detectable label. For example, such means may be a labeled specific binding agent as described herein. In general, such a “detectable label” may be any appropriate chemical substance or enzyme, which directly or indirectly generates a detectable compound or signal in a chemical, physical, optical, or enzymatic reaction. For example, a fluorescent or radioactive label can be used to generate fluorescence or x-rays as detectable signal. A dye can be used to generate an optical signal. Alkaline phosphatase, horseradish peroxidase and β-galactosidase are examples of enzyme labels (and at the same time optical labels) which catalyze the formation of chromogenic reaction products. Preferably, the label does not negatively affect the characteristics of the binding partner to which the label is conjugated. A preferred detectable label is an optically detectable label, such as a chromophore or a fluorescent label.

The present invention is further characterized by the following items:

Item 1. A method for detecting a trichothecene effect comprising: (c) determining in a test sample that is a blood sample the expression level of at least one miRNA having at least 90% sequence identity, preferably having at least 95% sequence identity, preferably being identical to a sequence selected from the group consisting of SEQ ID NO: 1 (ssc-miR-205), SEQ ID NO: 2 (ssc-miR-128), SEQ ID NO: 3 (ssc-miR-16), SEQ ID NO: 4 (ssc-miR-451), SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p); and (d) comparing the expression level with a reference value.

Item 2. The method of item 1, wherein a deviation in the expression level of said at least one miRNA in the test sample in comparison with the reference value is indicative for a trichothecene effect.

Item 3. The method of item 1 or 2 further comprising (e) detecting a trichothecene effect if the expression level of said at least one miRNA in the test sample deviates in comparison with the reference value.

Item 4. The method of any one of the preceding items, further comprising providing information on the test result of the method.

Item 5. The method of any one of the preceding items, wherein the reference value corresponds to the expression level of said at least one miRNA in a control sample.

Item 6. The method of any one of the preceding items, wherein the trichothecene effect is trichothecene exposure.

Item 7. The method of any one of the preceding items, wherein the trichothecene is a type B trichothecene.

Item 8. The method of any one of the preceding items, wherein the trichothecene is deoxynivalenol (DON) or a deoxynivalenol derivative.

Item 9. The method of any one of the preceding items, wherein the trichothecene is deoxynivalenol (DON).

Item 10. The method of any one of the preceding items, wherein the test sample is obtained from a subject.

Item 11. The method of any one of the preceding items, wherein the deviation comprises an increase and/or decrease of the expression level of at least one of said at least one miRNA.

Item 12. The method of any one of the preceding items, wherein the deviation is statistically significant.

Item 13. The method of any one of the preceding items, wherein the control sample has been obtained from a subject that has not been exposed to a trichothecene or is a mean value of multiple samples that have been obtained from one or more subjects that have not been exposed to a trichothecene.

Item 14. The method of any one of the preceding items wherein the test sample is a blood serum sample.

Item 15. The method of any one of the preceding items, wherein the at least one miRNA is selected from a miRNA having at least 90% sequence identity, preferably having at least 95% sequence identity, preferably being identical to a sequence selected from the group consisting of SEQ ID NO: 1 (ssc-miR-205), SEQ ID NO: 2 (ssc-miR-128), SEQ ID NO: 3 (ssc-miR-16), and SEQ ID NO: 4 (ssc-miR-451), and wherein an increase in the expression level in comparison with the reference value is indicative for a trichothecene effect.

Item 16. The method of any one of the preceding items, wherein the at least one miRNA is selected from a miRNA having at least 90% sequence identity, preferably having at least 95% sequence identity, preferably being identical to a sequence selected from the group consisting of SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p), and wherein a decrease in the expression level in comparison with the reference value is indicative for a trichothecene effect.

Item 17. The method of any one of the preceding items, wherein the at least one miRNA comprises two miRNA having at least 90% sequence identity, preferably having at least 95% sequence identity, preferably being identical to a sequence selected from the group consisting of SEQ ID NO: 1 (ssc-miR-205), SEQ ID NO: 2 (ssc-miR-128), SEQ ID NO: 3 (ssc-miR-16), SEQ ID NO: 4 (ssc-miR-451), SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p).

Item 18. The method any one of the preceding items, wherein the at least one miRNA comprises one or two of: (i) a miRNA having at least 90% sequence identity, preferably having at least 95% sequence identity, preferably being identical to SEQ ID NO: 1 (ssc-miR-205) and (ii) a miRNA having at least 90% sequence identity, preferably having at least 95% sequence identity, preferably being identical to SEQ ID NO: 2 (ssc-miR-128).

Item 19. The method any one of the preceding items, wherein the at least one miRNA comprises a miRNA having at least 90% sequence identity, preferably having at least 95% sequence identity, preferably being identical to SEQ ID NO: 1 (ssc-miR-205).

Item 20. The method any one of the preceding items, wherein the at least one miRNA comprises a miRNA having at least 90% sequence identity, preferably having at least 95% sequence identity, preferably being identical to SEQ ID NO: 2 (ssc-miR-128).

Item 21. The method of any one of the preceding items, wherein the test sample is obtained from a subject that is a mammal, an avian, or a fish.

Item 22. The method of item 21, wherein the subject is not a mouse.

Item 23. The method of item 21, wherein the subject is a farm animal.

Item 24. The method of item 21, wherein the subject is selected from the group consisting of pig, human, cow, horse, dog, chicken, and fish.

Item 25. The method of any one of the preceding items, the test sample is obtained from a subject of the genus Sus.

Item 26. The method of the preceding items, the test sample is obtained from a subject of the species Sus scrofa.

Item 27. The method of any one of the preceding items further comprising (b) providing the test sample.

Item 28. The method of any one of the preceding items, wherein the method comprises prior to obtaining the test sample from the subject (a) feeding the subject with feed suspected to contain a trichothecene.

Item 29. The method of any one of the preceding items, wherein the method is not for diagnosis.

Item 30. The method of any one of the preceding items, wherein the method is for detection of trichothecene in feed or food.

Item 31. The method of any one of items 1 to 28, wherein the method is for diagnosis.

Item 32. The method of item 31, wherein the method is for diagnosis of trichothecene exposure.

Item 33. The method of any one of items 1 to 30, wherein the method is for identifying feed for neutralization of trichothecene.

Item 34. The method of item 33, wherein neutralization comprises contacting the feed with a trichothecene neutralizing agent or applying a trichothecene neutralizing method.

Item 35. The method of item 34, wherein the trichothecene neutralizing agent is selected from the group consisting of one or more polypeptide(s), one or more microorganism(s), and one or more trichothecene-binding agent(s).

Item 36. The method of any one of items 33 to 35, comprising (a) feeding the subject with feed suspected to contain a trichothecene prior to obtaining the test sample from the subject; and/or (f) selecting the feed for neutralization of trichothecene if a deviation in the expression levels of the at least one miRNA in the test sample in comparison with the reference value is detected.

Item 37. The method of any one of items 33 to 36, further comprising providing the trichothecene neutralizing agent.

Item 38. The method of any one of items 33 to 37, further comprising contacting the feed or food identified by the method with the trichothecene neutralizing agent or applying the trichothecene neutralizing method to the feed.

Item 39. The method of any one of items 1 to 30, wherein the method is for assessment of the capacity of a method or test compound to neutralize trichothecene.

Item 40. The method of item 39, wherein the test compound is selected from the group consisting of one or more polypeptide(s), one or more microorganism(s) and one or trichothecene-binding agent(s).

Item 41. The method of item 39 or 40, wherein the method comprises prior to obtaining the test sample from the subject (a3) feeding a feed contacted with a trichothecene and the test compound to the subject.

Item 42. The method of item 41, wherein the method comprises prior to (a3) steps of: (a1) contacting the feed with a trichothecene, and (a2) contacting the feed contacted with a trichothecene with the test compound; or (a1′) contacting the feed with the test compound, and (a2′) contacting the feed contacted with the test compound with a trichothecene; or (a1″) contacting a trichothecene with the test compound, and (a2″) contacting the trichothecene contacted with the test compound with the feed.

Item 43. The method of any one of items 39 to 42, wherein reference value is obtained from a control sample that is obtained from a subject that has been fed with a control feed.

Item 44. The method of item 43, wherein the control feed is essentially free of trichothecene.

Item 45. The method of item 44, wherein the presence of a deviation in the expression levels of the at least one miRNA between the test sample and the control sample is indicative for the capacity of the test compound to neutralize trichothecene.

Item 46. The method of item 43, wherein the control feed has been contacted with a trichothecene.

Item 47. The method of item 46, wherein the absence of a deviation in the expression levels of the at least one miRNA between the test sample and the positive control sample is indicative for the capacity of the test compound to neutralize trichothecene.

Item 48. The method of item 33 to 47, wherein neutralization is by conversion.

Item 49. The method of item 33 to 47, wherein neutralization is by binding.

Item 50. Use of at least one miRNA having at least 90% sequence identity, preferably having at least 95% sequence identity, preferably being identical to a sequence selected from the group consisting of SEQ ID NO: 1 (ssc-miR-205), SEQ ID NO: 2 (ssc-miR-128), SEQ ID NO: 3 (ssc-miR-16), SEQ ID NO: 4 (ssc-miR-451), SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p) for detecting a trichothecene effect in a test sample that is a blood sample.

Item 51. The use of item 50, wherein the use is for detecting a trichothecene exposure of a subject.

Item 52. A data processing system comprising a processor configured to perform a method comprising the steps of
(a) comparing the expression level of at least one miRNA having at least 90% sequence identity, preferably having at least 95% sequence identity, preferably being identical to a sequence selected from the group consisting of SEQ ID NO: 1 (ssc-miR-205), SEQ ID NO: 2 (ssc-miR-128), SEQ ID NO: 3 (ssc-miR-16), SEQ ID NO: 4 (ssc-miR-451), SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p) determined in a test sample that is a blood sample obtained from a subject with a reference value;
(b) determining a deviation, wherein the reference value corresponds to the expression level that has been determined in a control sample that has been obtained from a subject that has not been exposed to a trichothecene; or determining no deviation, wherein the reference value corresponds to the expression level that has been determined in a control sample that has been obtained from a subject that has been exposed to a trichothecene; and
(c) indicating whether a trichothecene effect is determined based on (b).

Item 53. A sequencing device capable of determining the level of at least one miRNA having at least 90% sequence identity, preferably having at least 95% sequence identity, preferably being identical to a sequence selected from the group consisting of SEQ ID NO: 1 (ssc-miR-205), SEQ ID NO: 2 (ssc-miR-128), SEQ ID NO: 3 (ssc-miR-16), SEQ ID NO: 4 (ssc-miR-451), SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p), comprising the data processing system of item 52.

Item 54. A computer program comprising instructions to cause the data processing system of item 52 or the sequencing device of item 53 to execute the steps of
(a) comparing the expression level of at least one miRNA having at least 90% sequence identity, preferably having at least 95% sequence identity, preferably being identical to a sequence selected from the group consisting of SEQ ID NO: 1 (ssc-miR-205), SEQ ID NO: 2 (ssc-miR-128), SEQ ID NO: 3 (ssc-miR-16), SEQ ID NO: 4 (ssc-miR-451), SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p) determined in a test sample that is a blood sample obtained from a subject with a reference value;
(b) determining a deviation, wherein the reference value corresponds to the expression level that has been determined in a control sample that has been obtained from a subject that has not been exposed to a trichothecene; or determining no deviation, wherein the reference value corresponds to the expression level that has been determined in a control sample that has been obtained from a subject that has been exposed to a trichothecene; and
(c) indicating whether a trichothecene effect is determined based on (b).

Item 55. A computer-readable medium having stored thereon the computer program of item 54.

Item 56. A kit for performing the method of any one of items 1 to 49 comprising a specific binding agent for at least one miRNA having at least 90% sequence identity, preferably having at least 95% sequence identity, preferably being identical to a sequence selected from the group consisting of SEQ ID NO: 1 (ssc-miR-205), SEQ ID NO: 2 (ssc-miR-128), SEQ ID NO: 3 (ssc-miR-16), SEQ ID NO: 4 (ssc-miR-451), SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p) comprised in or conjugated to a solid support.

Item 57. The kit of item 56, wherein the kit comprises a specific binding agent for up to 23 different miRNAs.

Item 58. The kit of item 56 or 57, wherein the specific binding agent is a nucleic acid, an antibody, an antigen binding fragment of an antibody, or a proteinaceous molecule having antibody-like properties.

Item 59. The kit of any one of items 56 to 58, further comprising means for detecting a miRNA that is bound to the specific binding agent.

Item 60. The kit of item 59, wherein the means for detecting a miRNA that is bound to the specific binding agent comprises a probe conjugated to a detectable label.

Item 61. The kit of item 60, wherein the detectable label is an optically detectable label.

Item 62. The kit of any one of items 56 to 61, wherein the kit comprises an ELISA assay kit.

Item 63. The kit of any one of items 56 to 61, wherein the kit comprises a lateral flow assay kit.

It must be noted that as used herein, the singular forms “a”, “an” and “the” include plural references and vice versa unless the context clearly indicates otherwise. Thus, for example, a reference to “a miRNA” or “a process” includes one or more of such miRNAs or processes, respectively, and a reference to “the process” includes equivalent steps and processes that could be modified or substituted known to those of ordinary skill in the art. Similarly, for example, a reference to “miRNAs” includes “a miRNA”.

Unless otherwise indicated, the term “at least” preceding a series of elements is to be understood to refer to every element in the series.

Those skilled in the art will recognize, or be able to ascertain using no more than routine experimentation, many equivalents to the specific embodiments of the invention described herein. Such equivalents are intended to be encompassed by the present invention.

The term “more than” includes the concrete number. For example, “more than 20” means ≥20.

Throughout this specification and the claims or items, unless the context requires otherwise, the word “comprise” and variations such as “comprises” and “comprising” will be understood to imply the inclusion of a stated integer (or step) or group of integers (or steps). It does not exclude any other integer (or step) or group of integers (or steps). When used herein, the term “comprising” can be substituted with “containing”, “composed of”, “including”, “having” or “carrying.”

The term “about” or “approximately” as used herein means within 20%, preferably within 10%, and more preferably within 5% of a given value or range. It includes, however, also the concrete number, e.g., about 20 includes 20.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms. The possibility to replace terms with each other is not to be understood that these terms are necessarily synonymous.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent applications, scientific publications, manufacturer's specifications, instructions, etc.) are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

### EXAMPLES

### Example 1: Animal Trial

### Material & Methods

### Feeding Trial

In total 60 female weaned pigs (approximately 4 weeks old, average weight 7.4 kg) were allocated to six different treatment groups (n=10). After an acclimatization period of 8 days, piglets received diets containing different levels of mycotoxins for 27 days. From day 28-36, all treatment groups received control diet (no mycotoxins added).

TABLE 1
Treatment groups in the proof-of-principle trial

| Group | | |
|---|---|---|
| number | Group name | Deoxynivalenol (ppb) |
| 1 | Control | <50 |
| 2 | DON low | 870 |
| 3 | DON high | 2493 |

On day 0, 7, 14, 21, 26, 32 and 35/36, serum samples were collected from individual animals. Serum samples were stored at −80° C. until analysis. On day 27/28, 5 animals per group were euthanized and tissue samples from jejunum and liver were collected. Tissue samples were stored in RNAlater buffer (ThermoFischer, USA, #AM7020) at 4° C. for 24 hours, and thereafter stored at −80° C. until further analysis. On day 35/36, the remaining piglets were euthanized and tissues were collected as described for day 27/28.

miRNA-Sequencing Jejunum and Liver

Jejunum and liver samples from the Control, DON low and DON high group collected on day 27/28 were subjected to small RNA sequencing (n=5 per group/tissue). To this end, approximately 20-25 mg of tissue were disrupted via a bead-beating step, and the total RNA, including RNA from approximately 18 nucleotides upwards, was extracted and purified with the miRNeasy Mini Kit (Qiagen, Germany) according to the manufacturer's recommendation. The concentration of isolated RNAs was estimated on a NanoDrop 2000 spectrophotometer (Thermo Scientific, USA), and RNA quality (RIN) with an Agilent TapeStation 4200 (Agilent Technologies, Germany). In total, 1 μg of total RNA was used for construction of small RNA sequencing libraries with the NEBNext Multiplex Small RNA Library preparation set for Illumina (New England Biolabs Inc, USA). Adapter ligated total RNA was reverse transcribed into cDNA, which served as template for PCR amplification (15 cycles) using Illumina's SR Primer and Index Primers 1-18. Barcoded DNA libraries were purified using the QIAQuick PCR purification protocol (Qiagen, Germany) and quantified by DNA 1000 high sensitivity chip (Agilent Technologies, Germany). Purified samples were pooled at equimolar (50 nM) concentration. Size selection was performed using gel purification to select for insert sizes between 18 and 50 nucleotides. The final multiplexed libraries were sequenced on a NextSeq 500 sequencing instrument according to the manufacturer's instructions (HiSeq 2500 Lane Run, 50 pb cycle, single end, 6.5 mio reads/sample). Raw data were de-multiplexed and FASTQ files for each sample were generated using the bcl2fastq software (Illumina inc.). FASTQ data were checked using the FastQC tool (http://www.bioinformatics.babraham.ac.uk/projects/fastqc/).

Sequencing data were processed in-house. The quality of the sequences was assessed with FastQC. Adaptors were trimmed using BBDuk from the BBMap toolkit v38.22 (minlen=18 maxlen=25 qtrim=rl trimq=20 ktrim=r k=21 mink=11 hdist=2). For microRNA identification, the trimmed and quality filtered reads were aligned with bowtie v1.2.2 (Langmead et al, 2009, Genome Biol 10:R25) to the miRbase v21 (http://www.mirbase.org/) (Kozomara & Griffiths-Jones, 2014, Nucleic Acids Research 42(D1):D68-D73), specifically to the microRNAs of Sus scrofa (genome assembly 10.2). A count table of reads mapping to each microRNA was used for downstream analyses. Data evaluation was performed in-house and comprised exploratory expression analysis (e.g. via principal component analysis, non-negative matrix factorization or co-expression modules identification) using normalized read counts by the read counts per library. Raw reads were used for differential expression analysis (via DESeq2 package in R, Love et al, 2014, Genome Biology 15: 550). The p-values obtained were adjusted for multiple testing using Benjamini-Hochberg false discovery rate (FDR). Adjusted p-values of p<0.1 (FDR 10%) were considered significant.

miRNA-Sequencing Serum

Serum samples from the Control, DON low and DON high group collected on day 26 were subjected to small RNA sequencing (n=5 per group, same individuals as for sequencing of jejunum/liver samples). To this end, total RNA was extracted from 15 pig serum samples using the miRNeasy Mini Kit (Cat #217004, Qiagen, Germany). Samples are thawed at RT and centrifuged at 12,000 g for 5 minutes to remove any cellular debris. For each sample, 200 μL of serum were homogenized with 1000 μL Qiazol. A synthetic RNA oligonucleotide mix obtained from the miRCURY Spike-In kit (Cat #339390, Qiagen, Germany) was added to each sample at equimolar amounts prior to RNA extraction. These spike-ins can be subsequently used to monitor RNA extraction efficiency. Following incubation at room temperature for 15 minutes, 200 μL chloroform were added to the lysates followed by cooled centrifugation at 12,000 g for 15 minutes at 4° C. Precisely 600 μL of the upper aqueous phase are mixed with 7 μL glycogen (5 mg/mL) to enhance precipitation. Samples were transferred to a miRNeasy mini column, and RNA is precipitated with ethanol followed by automated washing with RPE and RWT buffer in a QiaCube liquid handling robot. Finally, total RNA was eluted in 30 L nuclease free water and stored at −80° C. until further analysis. Equal volumes of total RNA (2 μl) were used for small RNA library preparation using the CleanTag Small RNA Library Preparation Kit (Cat #L-3206-24, TriLink Biotechnologies, USA). Adapter-ligated libraries were amplified (23 PCR cycles) using barcoded Illumina reverse primers in combination with the Illumina forward primer. A pool of 15 small RNA sequencing libraries was prepared at equimolar rates on the basis of a DNA-1000 bioanalyzer results (Agilent, CA). Sequencing was performed on an Illumina HiSeq 2500 with 50 bp single-end reads.

Sequencing data were processed in-house. The quality of the sequences was assessed with FastQC. Adaptors were trimmed using BBDuk from the BBMap toolkit v38.22 (minlen=18 maxlen=25 qtrim=rl trimq=20 ktrim=r k=21 mink=11 hdist=2). For microRNA identification, the trimmed and quality filtered reads were aligned with bowtie v1.2.2 (Langmead et al, 2009, Genome Biol 10:R25) to the miRbase v21 (http://www.mirbase.org/) (Kozomara & Griffiths-Jones, 2014, Nucleic Acids Research 42(D1):D68-D73), specifically to the microRNAs of Sus scrofa (genome assembly 10.2). A count table of reads mapping to each microRNA was used for downstream analyses. Data evaluation was performed in-house and comprised exploratory expression analysis (e.g. via principal component analysis and co-expression modules identification) using normalized read counts by the read counts per library. Raw reads were used for differential expression analysis (via DESeq2 package in R, Love et al, 2014, Genome Biology 15: 550). The p-values obtained were adjusted for multiple testing using Benjamini-Hochberg false discovery rate (FDR). Adjusted p-values of p<0.1 (FDR 10%) were considered significant.

Quantitative Real-Time PCR (qPCR) miRNA Analysis Serum

Prior to qPCR analysis of miRNAs in serum, samples were analyzed for hemolysis. Serum samples from the Control, DON low and DON high group collected on d26 were subjected to targeted analysis of ssc-miR-16, ssc-miR-128, ssc-miR-205 and ssc-miR-451.

Following centrifugation at 12,000 g for 5 min to remove any cellular debris, 200 μL of serum were mixed with 1000 μL Qiazol and 1 μL synthetic Spike-Ins (Qiagen, Germany), and RNA was isolated using the miRNeasy Mini Kit (Qiagen, Germany). Following incubation at room temperature for 15 minutes, 200 μL chloroform were added to the lysates followed by cooled centrifugation at 12,000 g for 15 minutes at 4° C. Precisely 600 μL of the upper aqueous phase are mixed with 7 μL glycogen (5 mg/mL) to enhance precipitation. Samples were transferred to a miRNeasy mini column, and RNA was precipitated with ethanol followed by automated washing with RPE and RWT buffer in a QiaCube liquid handling robot. Finally, total RNA was eluted in 30 μL nuclease free water and stored at −80° C. until further analysis. We synthesized cDNA using the miRCury LNA RT Kit (Qiagen, Germany; Table 2). In total, 2 μL of purified RNA were used per 10 μL RT reaction, to which a defined molar amount of the non-mammalian microRNA cel-miR-39 was added to be used cDNA Spike-In. Real-time PCR amplification was performed in a 96-well plate format in a Roche LC480 II instrument (Roche, Germany) and miRCURY SYBR® Green mastermix (Qiagen, Germany) with the following settings: 95° C. for 10 min, 45 cycles of 95° C. for 10 s and 60° C. for 60 s, followed by melting curve analysis. To calculate the cycle of quantification values (Cq-values), the second derivative method was used. Cq-values were normalized to UniSp4 exogenous Spike-In by subtracting the individual microRNA Cq-value from the Cq-value of UniSp4 for that sample.

To remove any baseline differences in the miRNA abundances, the Cq-values were further normalized against the do values. To this end, for each sample the individual miRNA Cq-values of d26 were subtracted from the Cq-value of do. Statistical analysis was performed by comparing the Cq-values between the treatment groups for each sampling day. P-values were calculated with the Wilcoxon test in R.

### Results

### Feeding Trial

The different treatment diets had no statistically significant effect on body weight or body weight gain.

miRNA-Sequencing Jejunum and Liver

Overall, DON did not exhibit a strong effect on the miRNA expression profiles in liver and jejunum, respectively, and no treatment-dependent clustering was observed. In line with that, no differentially expressed miRNAs were found in tissues when comparing the Control and DON low group (with a FDR<10%, which corresponds to an adjusted p-value<0.1). Yet, one differentially expressed miRNA, namely ssc-miR-10b, was identified in liver when comparing samples of the Control and DON high group.

miRNA-Sequencing Serum

Surprisingly, differential abundance analysis showed that 15 (DON low vs Control) and 16 (DON high vs Control) miRNAs were significantly altered after DON exposure (FDR<10%, Table 2). In total, eight miRNAs were significantly altered in both DON low and DON high (Table 2, highlighted in bold). Out of those, four miRNAs were down-regulated upon DON treatment (ssc-miR-10b, ssc-miR-99b, ssc-miR-192, ssc-miR-374a-3p), and four were up-regulated in DON exposed pigs (ssc-miR-16, ssc-miR-128, ssc-miR-205, ssc-miR-451). The sequences of those eight microRNAs are provided in Table 3.

When comparing results to miRNA-sequencing data from liver, no direct correlation was observed. Still, ssc-miR-10b was significantly decreased in both matrices.

TABLE 2
Log2 fold changes and adjusted p-values of miRNAs significantly
affected in the DON low or DON high group compare to the Control.
The eight miRNAs affected both in DON low and DON high are indicated
in bold. n.s., not significant (adjusted p-value > 0.1).

| | | Log2 fold vs Control | | Adjusted p-value vs control | |
|---|---|---|---|---|---|
| | MicroRNA ID | DON low | DON high | DON low | DON high |
| Up-regulated | ssc-miR-16 | 1.217 | 0.773 | 0.082 | 0.067 |
| microRNAs | ssc-miR-17-5p | n.s. | 1.194 | n.s. | 0.053 |
| | ssc-miR-92a | n.s. | 0.476 | n.s. | 0.067 |
| | ssc-miR-128 | 1.124 | 0.887 | 0.070 | 0.053 |
| | ssc-miR-148a-3p | n.s. | 0.521 | n.s. | 0.014 |
| | ssc-miR-205 | 5.774 | 5.102 | 0.059 | 0.032 |
| | ssc-miR-339 | 1.345 | n.s. | 0.082 | n.s. |
| | ssc-miR-339-5p | 1.346 | n.s. | 0.082 | n.s. |
| | ssc-miR-361-3p | 2.023 | n.s. | 0.082 | n.s. |
| | ssc-miR-451 | 1.375 | 0.808 | 0.073 | 0.052 |
| Down-regulated | ssc-miR-10b | −1.129 | −0.754 | 0.059 | 0.033 |
| microRNAs | ssc-miR-10a-5p | −0.806 | n.s. | 0.082 | n.s. |
| | ssc-miR-92b-3p | −1.058 | n.s. | 0.082 | n.s. |
| | ssc-miR-99b | −1.393 | −1.226 | 0.070 | 0.013 |
| | ssc-miR-103 | n.s. | −0.630 | 0.013 | n.s. |
| | ssc-miR-107 | n.s. | −0.619 | 0.013 | n.s. |
| | ssc-miR-126-5p | −1.298 | n.s. | 0.059 | n.s. |
| | ssc-miR-192 | −1.133 | −0.928 | 0.082 | 0.017 |
| | ssc-miR-374a-3p | −1.271 | −1.443 | 0.070 | 0.016 |
| | ssc-miR-1388 | −1.281 | n.s. | 0.070 | n.s. |
| | ssc-let-7a | n.s. | −0.502 | n.s. | 0.097 |
| | ssc-let-7f-5p | n.s. | −0.503 | n.s. | 0.048 |
| | ssc-let-7g | n.s. | 0.662 | n.s. | 0.097 |

TABLE 3
Sequences of the eight miRNAs significantly affected both the DON high and DON
low group.

| miRNA ID | miRNA sequence | Alternative miRNA IDs |
|---|---|---|
| ssc-miR-16 | UAGCAGCACGUAAAUAUUGGC | hsa-miR-16-5p, age-miR-16, cfa- |
| | G (SEQ ID NO: 3) | miR-16, cgr-miR-16-5p, eca-miR- |
| | | 16, ggo-miR-16, lla-miR-16, mdo- |
| | | miR-16-5p, mml-miR-16-5p, |
| | | mmu-miR-16-5p, mne-miR-16, |
| | | oan-miR-16a-5p, ppa-miR-16, ptr- |
| | | miR-16, rno-miR-16-5p, sla-miR-16, |
| ssc-miR-128 | UCACAGUGAACCGGUCUCUUU | hsa-miR-128-3p, bta-miR-128, |
| | (SEQ ID NO: 2) | ccr-miR-128, cfa-miR-128, cgr- |
| | | miR-128-3p, eca-miR-128, gga- |
| | | miR-128-3p, ipu-miR-128, mdo- |
| | | miR-128a-3p, mdo-miR-128b-3p, |
| | | mml-miR-128b-3p, mmu-miR- |
| | | 128-3p, oan-miR-128-3p, ppy- |
| | | miR-128, ptr-miR-128, rno-miR- |
| | | 128-3p, sha-miR-128, tgu-miR- |
| | | 128-3p |
| ssc-mR-205 | UCCUUCAUUCCACCGGAGUCUG | hsa-miR-205-5p, age-miR-205, |
| | (SEQ ID NO: 1) | bta-miR-205, ccr-miR-205, cfa- |
| | | miR-205, eca-miR-205, fru-miR- |
| | | 205, gga-miR-205a, ggo-miR-205, |
| | | lla-miR-205, mdo-miR-205a, |
| | | mml-miR-205, mmu-miR-205-5p, |
| | | mne-miR-205, ppa-miR-205, ppy- |
| | | miR-205, ptr-miR-205, tni-miR- |
| | | 205, xla-miR-205, xtr-miR-205a |
| ssc-miR-451 | AAACCGUUACCAUUACUGAGU | hsa-miR-451a, cfa-miR-451, dre- |
| | U (SEQ ID NO: 4) | miR-451, mml-miR-451, mmu- |
| | | miR-451a, ppy-miR-451, ptr-miR- |
| | | 451, rno-miR-451-5p, xtr-miR- |
| | | 451 |
| ssc-miR-10b | UACCCUGUAGAACCGAAUUUG | gga-miR-10b-5p, cgr-miR-10b-5p, |
| | U (SEQ ID NO: 5) | oan-miR-10b-5p |
| ssc-miR-99b | CACCCGUAGAACCGACCUUGCG | hsa-miR-99b-5p, bta-miR-99b, |
| | (SEQ ID NO: 6) | cfa-miR-99b, eca-miR-99b, mml- |
| | | miR-99b-5p, mmu-miR-99b-5p, |
| | | ptr-miR-99b, rno-miR-99b-5p |
| ssc-miR-192 | CUGACCUAUGAAUUGACAGCC | hsa-miR-192-5p, cfa-miR-192, |
| | (SEQ ID NO: 7) | eca-miR-192, ggo-miR-192, mml- |
| | | miR-192-5p, mmu-miR-192-5p, |
| | | oan-miR-192-5p, ppy-miR-192, |
| | | ptr-miR-192, rno-miR-192-5p |
| ssc-miR-374a-3p | CUUAUCAGGUUGUAUUGUAAU | |
| | U (SEQ ID NO: 8) | |

qPCR Analysis Serum

qPCR analysis of the four up-regulated miRNAs was from samples taken at day 26 performed to validate small RNA sequencing results. Normalized Cq-value log 2 fold change for ssc-miR-16, ssc-miR-128, ssc-miR-205 and ssc-miR-451 were increased in the DON groups compared to the Control (Table 4).

TABLE 4
qPCR log2 fold changes of ssc-miR-16, ssc-miR-
128, ssc-miR-205 and ssc-miR-451 in the DON low
and DON high groups compare to the Control.

| miRNA ID | log2 fold change | Group |
|---|---|---|
| ssc-miR-451 | 1.835 | DON_high |
| ssc-miR-205 | 2.455 | DON_high |
| ssc-miR-128 | 1.879 | DON_high |
| ssc-miR-16 | 2.227 | DON_high |
| ssc-miR-451 | 2.663 | DON_low |
| ssc-miR-205 | 4.973 | DON_low |
| ssc-miR-128 | 3.885 | DON_low |
| ssc-miR-16 | 3.504 | DON_low |

The invention illustratively described herein may suitably be practiced in the absence of any element or elements, limitation or limitations, not specifically disclosed herein. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A method for detecting a trichothecene effect comprising: (c) determining in a test sample that is a blood sample the expression level of at least one miRNA having at least 90% sequence identity, preferably having at least 95% sequence identity, preferably being identical to a sequence selected from the group consisting of SEQ ID NO: 1 (ssc-miR-205), SEQ ID NO: 2 (ssc-miR-128), SEQ ID NO: 3 (ssc-miR-16), SEQ ID NO: 4 (ssc-miR-451), SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p); and (d) comparing the expression level with a reference value.

2. The method of claim 1 further comprising (e) detecting a trichothecene effect if the expression level of said at least one miRNA in the test sample deviates in comparison with the reference value.

3. The method of preceding claim 1 or 2, further comprising providing information on the test result of the method.

4. The method of any one of the preceding claims, wherein the trichothecene is a type B trichothecene.

5. The method of any one of the preceding claims, wherein the trichothecene is deoxynivalenol (DON) or a deoxynivalenol derivative.

6. The method any one of the preceding claims, wherein the at least one miRNA comprises one or two of:
(i) a miRNA having at least 90% sequence identity, preferably having at least 95% sequence identity, preferably being identical to SEQ ID NO: 1 (ssc-miR-205) and
(ii) a miRNA having at least 90% sequence identity, preferably having at least 95% sequence identity, preferably being identical to SEQ ID NO: 2 (ssc-miR-128).

7. The method of any one of the preceding claims, wherein the test sample is obtained from a subject that is a mammal, an avian, or a fish.

8. The method of the preceding claims, the test sample is obtained from a subject of the species Sus scrofa.

9. The method of any one of the preceding claims, wherein the method is for detection of trichothecene in feed or food.

10. Use of at least one miRNA having at least 90% sequence identity, preferably having at least 95% sequence identity, preferably being identical to a sequence selected from the group consisting of SEQ ID NO: 1 (ssc-miR-205), SEQ ID NO: 2 (ssc-miR-128), SEQ ID NO: 3 (ssc-miR-16), SEQ ID NO: 4 (ssc-miR-451), SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p) for detecting a trichothecene effect in a test sample that is a blood sample.

11. A data processing system comprising a processor configured to perform a method comprising the steps of
(a) comparing the expression level of at least one miRNA having at least 90% sequence identity, preferably having at least 95% sequence identity, preferably being identical to a sequence selected from the group consisting of SEQ ID NO: 1 (ssc-miR-205), SEQ ID NO: 2 (ssc-miR-128), SEQ ID NO: 3 (ssc-miR-16), SEQ ID NO: 4 (ssc-miR-451), SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p) determined in a test sample that is a blood sample obtained from a subject with a reference value;
(b) determining a deviation, wherein the reference value corresponds to the expression level that has been determined in a control sample that has been obtained from a subject that has not been exposed to a trichothecene; or
determining no deviation, wherein the reference value corresponds to the expression level that has been determined in a control sample that has been obtained from a subject that has been exposed to a trichothecene; and
(c) indicating whether a trichothecene effect is determined based on (b).

12. A sequencing device capable of determining the level of at least one miRNA having at least 90% sequence identity, preferably having at least 95% sequence identity, preferably being identical to a sequence selected from the group consisting of SEQ ID NO: 1 (ssc-miR-205), SEQ ID NO: 2 (ssc-miR-128), SEQ ID NO: 3 (ssc-miR-16), SEQ ID NO: 4 (ssc-miR-451), SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p), comprising the data processing system of claim 11.

13. A computer program comprising instructions to cause the data processing system of claim 11 or the sequencing device of claim 12 to execute the steps of
(a) comparing the expression level of at least one miRNA having at least 90% sequence identity, preferably having at least 95% sequence identity, preferably being identical to a sequence selected from the group consisting of SEQ ID NO: 1 (ssc-miR-205), SEQ ID NO: 2 (ssc-miR-128), SEQ ID NO: 3 (ssc-miR-16), SEQ ID NO: 4 (ssc-miR-451), SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p) determined in a test sample that is a blood sample obtained from a subject with a reference value;
(b) determining a deviation, wherein the reference value corresponds to the expression level that has been determined in a control sample that has been obtained from a subject that has not been exposed to a trichothecene; or
determining no deviation, wherein the reference value corresponds to the expression level that has been determined in a control sample that has been obtained from a subject that has been exposed to a trichothecene; and
(c) indicating whether a trichothecene effect is determined based on (b).

14. A computer-readable medium having stored thereon the computer program of claim 13.

15. A kit for performing the method of any one of claims 1 to 9 comprising a specific binding agent for at least one miRNA having at least 90% sequence identity, preferably having at least 95% sequence identity, preferably being identical to a sequence selected from the group consisting of SEQ ID NO: 1 (ssc-miR-205), SEQ ID NO: 2 (ssc-miR-128), SEQ ID NO: 3 (ssc-miR-16), SEQ ID NO: 4 (ssc-miR-451), SEQ ID NO: 5 (ssc-miR-10b), SEQ ID NO: 6 (ssc-miR-99b), SEQ ID NO: 7 (ssc-miR-192), and SEQ ID NO: 8 (ssc-miR-374a-3p) comprised in or conjugated to a solid support.
